(19) United States
(12) Patent Application Publication
(10) Pub. No.: US 2008/0051463 A1
(43) Pub. Date: Feb. 28, 2008

(54) ANTHRACENE COMPOUNDS AND THEIR USE FOR TREATING BENIGN AND MALIGNANT TUMOR DISORDERS

(75) Inventors: Matthias Gerlach, Brachttal (DE); Eckhard Gunther, Maintal (DE); Peter Schmidt, Schoeneck (DE); Helge Prinz, Havixbeck (DE); Konrad Bohm, Jena (DE); Eberhard Unger, Jena (DE)

Correspondence Address: OBLON, SPIVAK, MCCLELLAND MAIER & NEUSTADT, P.C., 1940 DUKE STREET, ALEXANDRIA, VA, 22314, US

(73) Assignee: AETERNA ZENTARIS GmbH, Frankfurt (DE)

(21) Appl. No.: 11/833,254

(22) Filed: Aug. 3, 2007

Related U.S. Application Data
(60) Provisional application No. 60/894,935, filed on Mar. 15, 2007.
(60) Provisional application No. 60/835,431, filed on Aug. 4, 2006.

(30) Foreign Application Priority Data
Aug. 7, 2006 (EP) 06016401.9

(51) Int. Cl. A61K 31/12; A61P 35/00; C07C 49/14

(52) U.S. Cl. 514/680; 568/326

(57) ABSTRACT

The present invention provides novel anthracene compounds according to the formulae (I) and (II) and also selected anthracene compound compounds. The present invention furthermore provides a process for preparing such anthracene compounds. Also provided are pharmaceutical formulations comprising these anthracene compounds. The anthracene compounds provided are particularly suitable for the treatment and/or prophylaxis of physiological and/or pathophysiological conditions which can be treated by inhibiting tubulin polymerization and/or by inhibiting microtubuli-based motor proteins, in particular various tumor disorders.

### REFERENCE TO PRIOR APPLICATIONS

This application claims priority to U.S. provisional application U.S. 60/835,431 filed Aug. 4, 2006, and U.S. 60/894,935 filed Mar. 15, 2007, as well as to EP 06016401.9 filed Aug. 7, 2006, all incorporated herein by reference.

### TECHNICAL FIELD

The invention relates to novel anthracene compounds and to methods for treating benign and malignant tumor disorders. These compounds act as inhibitors of tubulin polymerization and/or as inhibitors of microtubuli-based motor proteins, in particular kinesins and dyneins, and are suitable for the treatment or prophylaxis of physiological and/or pathophysiological conditions which can be treated by inhibiting tubulin polymerization and/or by inhibiting microtubuli-based motor proteins, and also diverse tumor disorders.

### STATE OF THE ART

For the next few years, a dramatic increase in oncoses and tumor-related deaths is expected worldwide. In 2001, worldwide approximately 10 million people were suffering from cancer and over 6 million people died from this disease. The development of tumors is a fundamental disease of higher organisms in the plant kingdom, in the animal kingdom and in humans. The generally recognized multistep model of carcinogenesis assumes that as a result of the accumulation of a number of mutations in an individual cell it is so modified in its proliferation and differentiation behaviour that finally, via benign intermediate stages, a malignant state with metastasis is reached. Behind the term cancer or tumor, a clinical picture with more than 200 different individual diseases is hidden. Oncoses can proceed in a benign or malignant manner. The most important tumors are those of the lung, the breast, the stomach, the cervix, the prostate, the head and neck, the large and small intestine, the liver and the blood system. There are great differences with respect to course, prognosis and response to therapy. More than 90% of the cases recognized relate to solid tumors, which in particular in the advanced stages or on metastasis are at present treatable with difficulty or untreatable. The three pillars of cancer control are still surgical removal, irradiation and chemotherapy. In spite of great advances it has still not been possible to develop medicaments which bring about a marked prolongation of the survival time or even a complete cure in the widespread solid tumors. The problem of the development of resistance to clinical tumor therapeutics is also becoming an increasingly more frequent occurrence. It is therefore meaningful to invent novel medicaments for the control of cancer.

One possible approach to controlling oncoses is the inhibition of cell division (mitosis). To date, a large number of antimitotically acting medicaments have been found to be effective in clinical oncology. Examples of these are the alkaloids vincristine and vinblastine in the context of the treatment of leukaemia and Hodgkin's lymphoma, and also paclitaxel and docetaxel for the treatment of metastatic breast cancer, pulmonary and ovary carcinomas.

However, these substances have some serious side-effects, such as, for example, neurotoxicity (Quasthoff S et al., J. Neurol. 2002, 249: 9-17), and in some cases they also lead to multiple resistance (Gottesman M M et al., Nat. Rev. Cancer 2002, 2: 48-58; Dumontet C et al., J. Clin. Oncol. 1999, 17: 1061-1070; Sangrajrang S et al., Chemotherapy 2000, 46: 327-334).

A relatively recent area of research is involved in the study of microtubuli-based motor proteins. These include in particular the kinesins, which are involved in mitosis and the transport of organelles (Yildiz A et al., Trends Cell Biol. 2005, 15: 112-120). The kinesin superfamily is characterized in detail in Miki H et al. (Proc. Natl. Acad. Sci. USA 2001, 98: 7004-7011). Further relevant works dedicated to kinesins, their multifarious functions and first kinesin inhibitors are: Hirokawa N et al., Exp. Cell Res. 2004, 301: 50-59; Zhu C et al., Mol. Biol. Cell 2005, 16: 3187-3199; Sarli V et al., ChemMedChem 2006, 1: 293-298.

Farid and co-workers describe the competitive photoaddition of acetylenes to the C═C and C═O bonds of p-quinones. A commercial application, in particular as medicaments, is neither described nor suggested (Farid S et al., Tetrahedron Letters 1968, 9(39): 4147-4150).

DE 102 01 228 describes 10-benzylidene-9(10H)anthracenones as novel inhibitors of tubulin polymerization.

DE 102 53 720 describes 10-phenylimino-9(10H)anthracenones as novel inhibitors of tubulin polymerization.

DE 102 53 746 describes 10-benzylidene-1,8-dichloro-9(10H)anthracenones as novel inhibitors of tubulin polymerization.

WO 2004/007470 describes novel anthracene compounds and their use as medicaments. The compounds described may act by inhibiting tubulin polymerization.

EP 1 645 556 describes arylpiperazinebenzoylamide compounds which are inhibitors of tubulin polymerization and suitable for use as pharmaceutical agents.

WO 02/060872 describes synthesis methods for compounds which may be characterized in a screening as potential inhibitors of tubulin polymerization.

Bryce-Smith and co-workers describe the photoaddition of diphenylacetylene, cycloocta-1,3-diene and cyclooctene to anthraquinone. A commercial application, in particular as medicaments, is neither described nor suggested (Bryce-Smith D et al., Tetrahedron Letters 1968, 9(24): 2863-2866).

Polman and co-workers describe photochemical reactions of acetylenes. Aromatic ketones are photoadded to arylacetylene. A commercial application, in particular as medicaments, is neither described nor suggested (Polman H et al., Recueil des Travaux Chimiques des Pays-Bas 1973, 92(7): 845-854).

### DESCRIPTION OF THE INVENTION

Accordingly, it is an object of the present invention to provide novel substances suitable for treating oncoses.

In one aspect, the object of the invention was surprisingly achieved by providing novel anthracene compounds according to the formula (I) 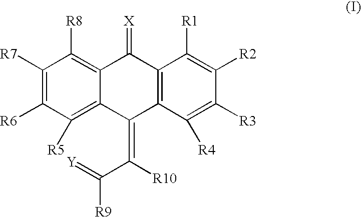
in which:
substituent X is independently selected from the group consisting of: “O, S, NR11, N—OR12, geminally attached hydrogen and hydroxyl”;
substituent Y is independently selected from the group consisting of: “O, S, NR13, N—OR14”;
substituents R1, R2, R3, R4, R5, R6, R7, R8 independently of one another are independently selected from the group consisting of: “hydrogen, unsubstituted or substituted alkyl, unsubstituted or substituted cycloalkyl, unsubstituted or substituted cycloalkyl-alkyl, unsubstituted or substituted heterocyclyl, unsubstituted or substituted heterocyclyl-alkyl, unsubstituted or substituted aryl, unsubstituted or substituted aryl-alkyl, unsubstituted or substituted heteroaryl, unsubstituted or substituted heteroaryl-alkyl, amino, mono-alkylamino, di-alkylamino, halogen, —F, —Cl, —Br, —I, alkyl substituted by one or more fluorine atoms, trifluoromethyl, cyano, straight-chain or branched cyano-alkyl, carbonyl, carboxyl, —COOH, alkoxycarbonyl, carboxy-alkyl, alkoxycarbonyl-alkyl, hydroxyl, alkoxy, aryl-alkoxy, benzyloxy, heteroaryl-alkoxy, alkoxycarbonylamino, alkoxycarbonylamino-alkyl”;
substituent R9 is independently selected from the group consisting of: “unsubstituted or substituted alkyl, unsubstituted or substituted cycloalkyl, unsubstituted or substituted cycloalkyl-alkyl, unsubstituted or substituted heterocyclyl, unsubstituted or substituted heterocyclyl-alkyl, unsubstituted or substituted aryl, unsubstituted or substituted aryl-alkyl, unsubstituted or substituted heteroaryl, unsubstituted or substituted heteroaryl-alkyl, —OR15, —NR16R17”;
substituent R10 is independently selected from the group consisting of: “hydrogen, unsubstituted or substituted alkyl, unsubstituted or substituted aryl, unsubstituted or substituted aryl-alkyl, unsubstituted or substituted heteroaryl, unsubstituted or substituted heteroaryl-alkyl”;
substituents R11, R12, R13, R14, R15, R16, R17 independently of one another are independently selected from the group consisting of: “hydrogen, unsubstituted or substituted alkyl, unsubstituted or substituted cycloalkyl, unsubstituted or substituted cycloalkyl-alkyl, unsubstituted or substituted heterocyclyl, unsubstituted or substituted heterocyclyl-alkyl, unsubstituted or substituted aryl, unsubstituted or substituted aryl-alkyl, unsubstituted or substituted heteroaryl, unsubstituted or substituted heteroaryl-alkyl”; with the proviso that the following compounds embraced by formula (I) are excluded:
(i) compound having the Chemical Abstract Service (CAS) Registry No. 262378-52-9 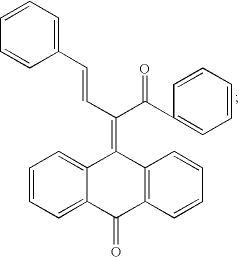
(ii) compound having the Chemical Abstract Service (CAS) Registry No. 121747-08-8 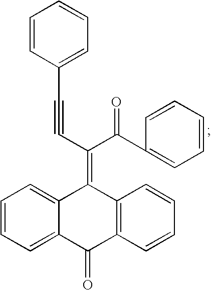
(iii) compound having the Chemical Abstract Service (CAS) Registry No. 63370-49-0 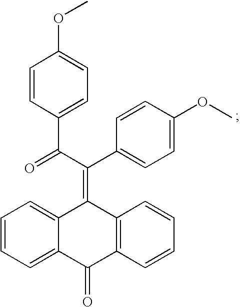
(iv) compound having the Chemical Abstract Service (CAS) Registry No. 19661-62-2 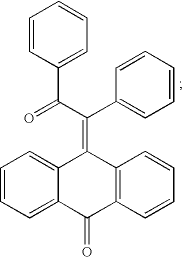
(v) compound having the Chemical Abstract Service (CAS) Registry No. 17665-75-7 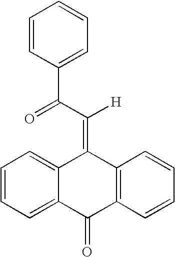
(vi) compound having the Chemical Abstract Service (CAS) Registry No. 83498-19-5 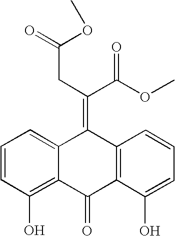
(vii) compound having the Chemical Abstract Service (CAS) Registry No. 42866-43-3 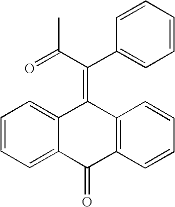
(viii) compound having the Beilstein Registry No. 3095926 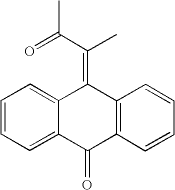
(ix) compound having the Chemical Abstract Service (CAS) Registry No. 42866-49-9

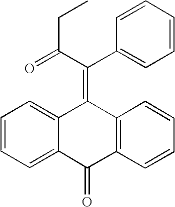

The substitution “geminally attached hydrogen and hydroxyl” in conjunction with the substituent X is to be understood in the context of the present invention as meaning that both “H” and “OH” are attached to one and the same carbon atom.

In a preferred embodiment, novel anthracene compound according to the formula (I) and according to the above substituent definition at the outset are provided

in which:

substituent X is independently selected from the group consisting of: “O, S, NH, geminally attached hydrogen and hydroxyl”;

substituent Y is independently “O or S”;

substituents R1, R2, R3, R4, R5, R6, R7, R8 are independently “hydrogen, fluorine, chlorine, bromine, iodine, hydroxyl, amino, nitro, cyano, methyl, trifluoromethyl”;

substituent R9 is independently selected from the group consisting of: “unsubstituted or substituted heterocyclyl, unsubstituted or substituted aryl, unsubstituted or substituted heteroaryl, —OR15, —NR16R17”;

substituent R10 is independently “hydrogen, alkyl”.

In a further preferred embodiment, novel anthracene compounds according to the formula (I) and according to the two above substituent definitions at the outset are provided

in which:

substituent R9 is independently selected from the group consisting of: “3,4-dimethoxy-phenyl; 3,4-dihydroxy-phenyl; 4-methoxy-thiophen-2-yl, thiomorpholin-4-yl; 4-methoxy-phenyl; 4-(4-methoxy-phenyl)-piperazin-1-yl, 2-hydroxy-3,4-dimethoxy-phenyl, 4-methylphenyl; 4-hydroxyphenyl; thiophen-2-yl; 3-hydroxyphenyl; 3-methoxy-phenyl; 2,6-dimethoxy-phenyl; 3,4,5-trimethoxy-phenyl; hydroxyl; methoxy; phenyl; 4-chloro-phenyl; 4-bromophenyl; 4-fluorophenyl; 3-chlorophenyl; 3-bromophenyl; 3-fluorophenyl; 4-fluoro-3-hydroxy-phenyl”.

A further preferred embodiment provides novel anthracene compounds selected from the group consisting of:

### 10-[2-(3,4-dihydroxy-phenyl)-2-oxo-ethylidene]-10H-anthracen-9-one (Compound 1)

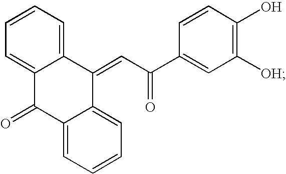

### 10-[2-(4-methoxy-thiophen-2-yl)-2-oxo-ethylidene]-10H-anthracen-9-one (Compound 2)

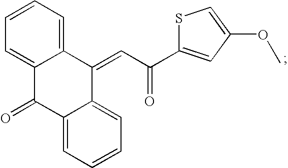

### 10-(2-oxo-2-thiomorpholin-4-ylethylidene)-10H-anthracen-9-one (Compound 3)

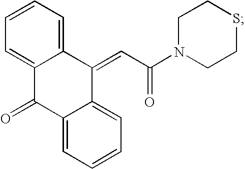

### 2-(10-hydroxy-10H-anthracen-9-ylidene)-1-(4-methoxy-phenyl)-ethanone (Compound 4)

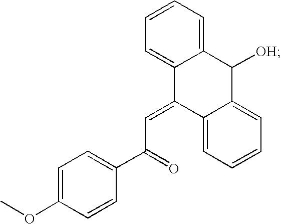

### 10-{2-[4-(4-methoxy-phenyl)-piperazin-1-yl]-2-oxo-ethylidene}-10H-anthracen-9-one (Compound 5)

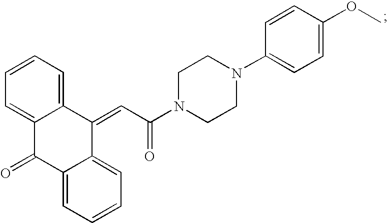

### 10-[2-(3,4-dimethoxy-phenyl)-2-oxo-ethylidene]-9(10H)-anthracenone (Compound 6)

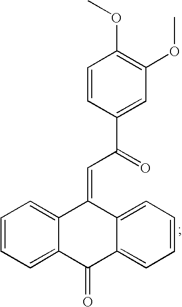

### 10-[2-(4-methoxy-phenyl)-2-oxo-ethylidene]-10H-anthracen-9-one (Compound 7)

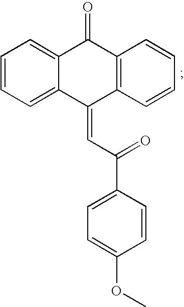

### 10-[2-(2-hydroxy-3,4-dimethoxy-phenyl)-2-oxo-ethylidene]-10H-anthracen-9-one (Compound 8)

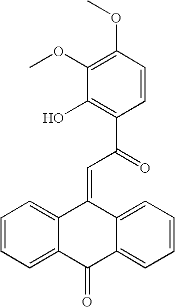

### 10-(2-oxo-2-p-tolyl-ethylidene)-10H-anthracen-9-one (Compound 9)

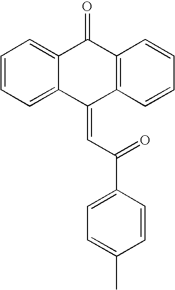

### 10-[2-(4-hydroxy-phenyl)-2-oxo-ethylidene]-10H-anthracen-9-one (Compound 10)

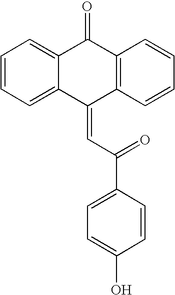

### 10-(2-oxo-2-thiophen-2-yl-ethylidene)-10H-anthracen-9-one (Compound 11)

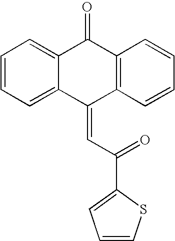

### 10-[2-(3-hydroxy-phenyl)-2-oxo-ethylidene]-10H-anthracen-9-one (Compound 12)

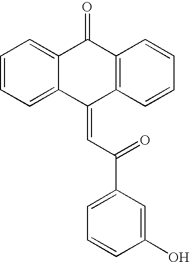

### 10-[2-(3-methoxy-phenyl)-2-oxo-ethylidne]-10H-anthracen-9-one (Compound 13)

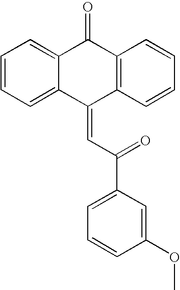

### (10-oxo-10H-anthracen-9-ylidene)-acetic acid 2,6-dimethoxy-phenyl ester (Compound 14)

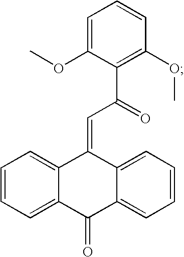

### (10-oxo-10H-anthracen-9-ylidene)-acetic acid methyl ester (Compound 15)

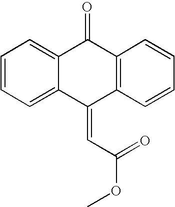

### 10-[2-oxo-2-(3,4,5-trimethoxy-phenyl)-ethylidene]-10H-anthracen-9-one (Compound 16)

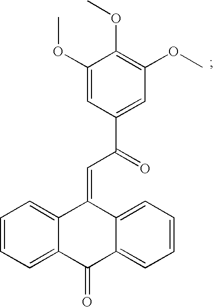

### (10-oxo-10H-anthracen-9-ylidene)-acetic acid (Compound 17)

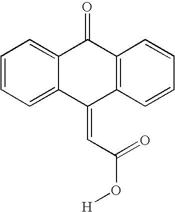

### 10-(2-oxo-2-phenyl-ethylidene)-10H-anthracen-9-one (Compound 18)

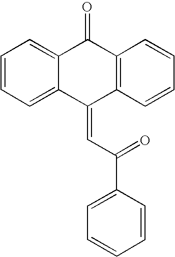

### 10-[2-(4-chloro-phenyl)-2-oxo-ethylidene]-10H-anthracen-9-one (Compound 19)

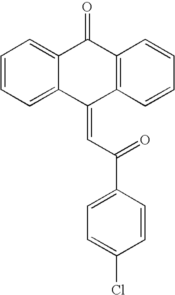

### 10-[2-(4-bromo-phenyl)-2-oxo-ethylidene]-10H-anthracen-9-one (Compound 20)

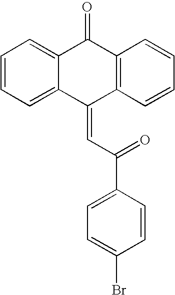

### 10-[2-(3-chloro-phenyl)-2-oxo-ethylidene]-10H-anthracen-9-one (Compound 21)

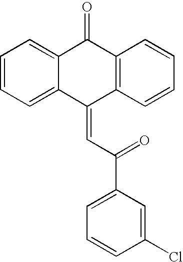

### 10-[2-(3-bromo-phenyl)-2-oxo-ethylidene]-10H-anthracen-9-one (Compound 22)

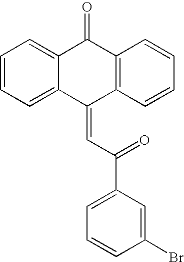

### 10-[2-(4-fluoro-phenyl)-2-oxo-ethylidene]-10H-anthracen-9-one (Compound 23)

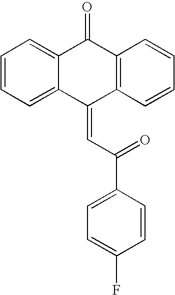

### 10-[2-(3-fluoro-phenyl)-2-oxo-ethylidene]-10H-anthracen-9-one (Compound 24)

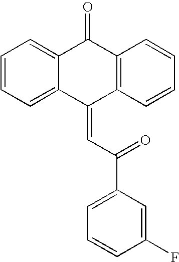

### 10-[2-(4-fluoro-3-hydroxy-phenyl)-2-oxo-ethylidene]-10H-anthracen-9-one (Compound 25)

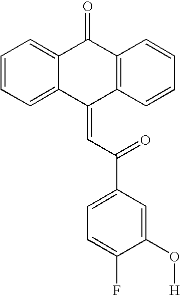

### 1,(3-4-dihydroxy-phenyl)-2-(10-thioxo-10H-anthracen-9-ylidene)-ethanone (Compound 26)

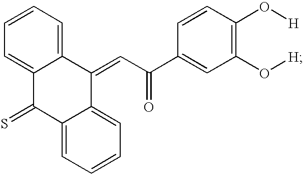

### 1-(3,4-dihydroxy-phenyl)-2-(10-imino-10H-anthracen-9-ylidene)-ethanone (Compound 27)

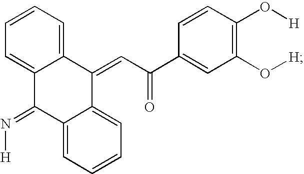

### 1-(3-hydroxy-phenyl)-2-(10-thioxo-10H-anthracen-9-ylidene)-ethanone (Compound 28)

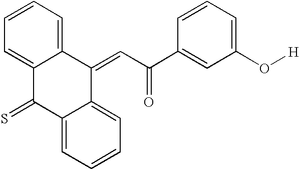

### 1-(3-hydroxy-phenyl)-2-(10-imino-10H-anthracen-9-ylidene)-ethanone (Compound 29)

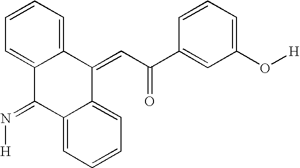

### 1-(4-fluoro-3-hydroxy-phenyl)-2-(10-thioxo-10H-anthracen-9-ylidene)-ethanone (Compound 30)

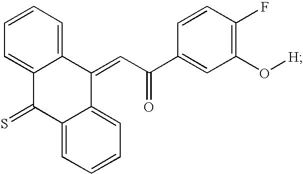

### 1-(4-fluoro-3-hydroxy-phenyl)-2-(10-imino-10H-anthracen-9-ylidene)-ethanone (Compound 31)

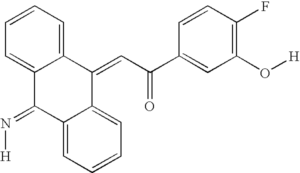

### 1,4-dichloro-10[2-(3-hydroxy-phenyl)-2-oxo-ethylidene]-10H-anthracen-9-one (Compound 32)

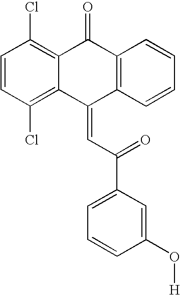

### 1,2-dichloro-10-[2-(3-hydroxy-phenyl)-2-oxo-ethylidene]-10H-anthracen-9-one (Compound 33)

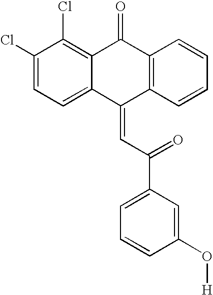

### 3,4-dichloro-10-[2-(3-hydroxy-phenyl)-2-oxo-ethylidene]-10H-anthracen-9-one (Compound 34)

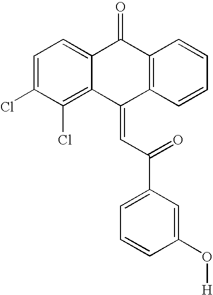

### 1,2-methyl-10-[2-(3-hydroxy-phenyl)-2-oxo-ethylidene]-10H-anthracen-9-one (Compound 35)

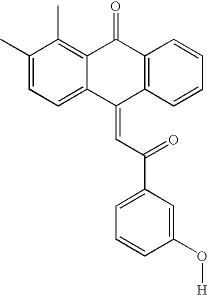

### 3-chloro-10-[2-(3-hydroxy-phenyl)-2-oxo-ethylidene]-10H-anthracen-9-one (Compound 36)

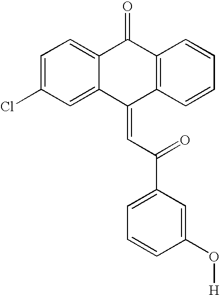

### 3-bromo-10-[2-(3-hydroxy-phenyl)-2-oxo-ethylidene]-10H-anthracen-9-one (Compound 37)

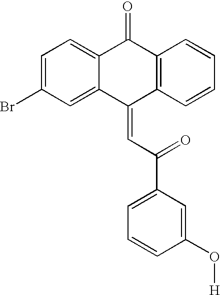

### 10-[2-(3-hydroxy-phenyl)-2-oxo-ethylidene]-3-methyl-10H-anthracen-9-one (Compound 38)

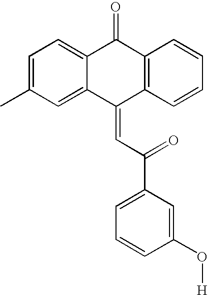

### 1,4-dichloro-10-[2-(3,4-dihydroxy-phenyl)-2-oxo-ethylidene]-10H-anthracen-9-one (Compound 39)

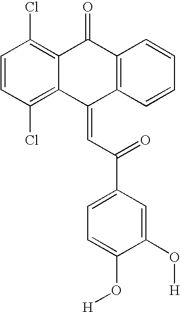

### 3,4-dimethyl-10-[2-(3-hydroxy-phenyl)-2-oxo-ethylidene]-10H-anthracen-9-one (Compound 40)

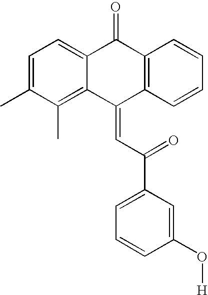

### 2-[-2-chloro-10-thioxo-10H-anthracen-9-ylidene]-1-(3-hydroxy-phenyl)-ethanone (Compound 41)

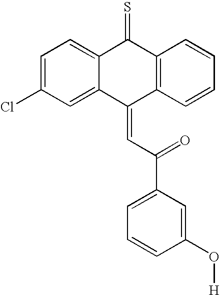

### 3-chloro-10-[2-(3,4-dihydroxy-phenyl)-2-oxo-ethylidene]-10H-anthracen-9-one (Compound 42)

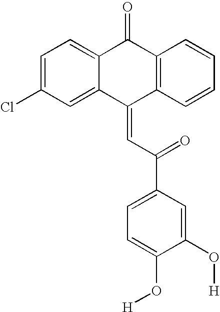

### 2-[2-chloro-10-imino-10H-anthracen-9-ylidene]-1-(3-hydroxy-phenyl)-ethanone (Compound 43)

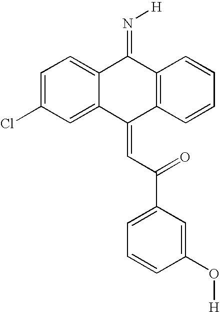

If, as a result of an error, chemical name and chemical structure of the compounds shown above do not correspond, the chemical structure of each individual compound—to prevent misunderstandings—is to be considered as being decisive for the unambiguous description of the compound.

Hereinbelow, novel anthracene compounds, shown above, of the generic formula (I), its two preferred generic embodiments, the explicitly shown anthracene compound compounds 1 to 43 and the anthracene compounds of the generic formula (II) and its two preferred generic embodiments are together referred to as “compounds according to the invention”.

The expressions and terms used for illustrating the compounds according to the invention have in principle, unless stated otherwise in the description or in the claims, the following meanings:

In the context of the present invention, the expression “alkyl” includes acyclic saturated or unsaturated hydrocarbon radicals, which may be branched or straight-chain and also unsubstituted or mono- or polysubstituted, having 1 to 20 carbon atoms, i.e. C₁-₂₀-alkanyls, C₂-₂₀-alkenyls and C₂-₂₀-alkynyls, preferably having 1 to 8 carbon atoms, i.e. C₁-₈-alkanyls, C₂-₈-alkenyls and C₂-₈-alkynyls. Here, alkenyls have at least one C—C double bond and alkynyls at least one C—C triple bond, it being possible for alkynyls additionally to also have at least one C—C double bond. Preferred alkyl radicals are methyl, ethyl, n-propyl, 2-propyl, n-butyl, sec-butyl, tert-butyl, n-pentyl, isopentyl, neo-pentyl, n-hexyl, 2-hexyl, n-heptyl, n-octyl, n-nonyl, n-decyl, n-undecyl, n-dodecyl, ethylenyl (vinyl), ethynyl, propenyl (—CH₂CH═CH₂; —CH═CH—CH₃, —C(═CH₂)—CH₃), propynyl (—CH₂—C≡CH, —C≡C—CH₃), butenyl, butynyl, pentenyl, pentynyl, hexenyl, hexynyl, heptenyl, heptynyl, octenyl, octadienyl and octynyl.

In the context of the present invention, the expression “cycloalkyl” refers to cyclic non-aromatic hydrocarbons having 1 to 3 rings having 3 to 20, preferably 3 to 12 carbon atoms, which hydrocarbons may be saturated or unsaturated, particularly preferably (C₃-C₈)-cycloalkyl. The cycloalkyl radical may also be part of a bi- or polycyclic system where, for example, the cycloalkyl radical is condensed with an aryl, heteroaryl or heterocyclyl radical as defined herein by any possible and desired ring member(s). Attachment to the compounds of the formulae (I) and (II) may be via any possible ring member of the cycloalkyl radical. Preferred cycloalkyl radicals are cyclopropyl, cyclobutyl, cyclopentyl, cyclohexyl, cycloheptyl and cyclooctyl, cyclodecyl, cyclohexenyl, cyclopentenyl and cyclooctadienyl.

The term “heterocyclyl” denotes a 3- to 14-membered, preferably 3-, 4-, 5-, 6-, 7- or 8-membered cyclic organic radical which contains at least 1, optionally 2, 3, 4 or 5, heteroatoms, in particular nitrogen, oxygen and/or sulphur, where the heteroatoms are identical or different and the cyclic radical is saturated or unsaturated, but not aromatic, and may be unsubstituted or mono- or polysubstituted. The heterocyclyl radical may also be part of a bi- or polycyclic system where, for example, the heterocyclyl radical is condensed with an aryl, heteroaryl or cycloalkyl radical as defined herein by any possible and desired ring member(s). The attachment to the compounds of the formulae (I) and (II) may be via any possible ring member of the heterocyclyl radical. Preferred heteroatoms are nitrogen, oxygen and sulphur. Preferred heterocyclyl radicals are tetrahydrofuryl, pyrrolidinyl, imidazolidinyl, thiazolidinyl, tetrahydropyranyl, piperidinyl, piperazinyl, morpholinyl, thiomorpholinyl, thiapyrrolidinyl, oxapiperazinyl, oxapiperidinyl and oxadiazolyl.

In the context of the present invention, the expression “aryl” refers to aromatic hydrocarbons having 3 to 14 carbon atoms, preferably 5 to 14 carbon atoms, particularly preferably having 6 to 14 carbon atoms, inter alia phenyls, naphthyls and anthracenyls. The aryl radical may also be part of a bi- or polycyclic system where, for example, the aryl radical is condensed with a heterocyclyl, heteroaryl or cycloalkyl radical as defined herein by any possible and desired ring member(s), for example with tetrahydrofuran, tetrahydrothiophene, pyrrolidine, imidazolidine, thiazolidine, tetrahydropyran, dihydropyran, piperidine, furan, thiophene, imidazole, thiazole, oxazole and isoxazole. The attachment to the compounds of the formulae (I) and (II) may be via any possible ring member of the aryl radical. Each aryl radical may be unsubstituted or mono- or polysubstituted, where the aryl substituents may be identical or different and may be in any possible position of the aryl. Preferred aryl radicals are phenyl, biphenyl, naphthyl and anthracenyl, but likewise indanyl, indenyl or 1,2,3,4-tetrahydronaphthyl.

The expression “heteroaryl” denotes a 5-, 6- or 7-membered cyclic aromatic radical which contains at least 1, optionally also 2, 3, 4 or 5, heteroatoms, in particular nitrogen, oxygen and/or sulphur, where the heteroatoms are identical or different and the heterocyclic may be unsubstituted or mono- or polysubstituted; in the case of substitution at the heterocycle, the heteroaryl substituents may be identical or different and may be in any possible position of the heteroaryl. The number of nitrogen atoms is preferably 0, 1, 2 or 3 and that of the oxygen and sulphur atoms is preferably 0 or 1. The heteroaryl radical may also be part of a bi- or polycyclic system where, for example, the heteroaryl radical is condensed with a heterocyclyl, aryl or cycloalkyl radical as defined herein by any possible and desired ring member(s). The attachment to the compounds of the formulae (I) and (II) may be via any possible ring member of the heteroaryl radical. The heterocycle may also be part of a bi- or polycyclic system. Preferred heteroatoms are nitrogen, oxygen and sulphur. Preferred heteroaryl radicals are pyrrolyl, furyl, thienyl, thiazolyl, isothiazolyl, oxazolyl, oxadiazolyl, isoxazolyl, pyrazolyl, imidazolyl, triazole, tetrazole, pyridinyl, pyrimidinyl, pyridazinyl, pyrazinyl, triazine, phthalazinyl, indolyl, indazolyl, indolizinyl, quinolinyl, isoquinolinyl, quinoxalinyl, quinazolinyl, pteridinyl, carbazolyl, phenazinyl, phenoxazinyl, phenothiazinyl and acridinyl.

For the purpose of the present invention, the expressions “alkyl-cycloalkyl”, “cycloalkylalkyl”, “alkyl-heterocyclyl”, “heterocyclylalkyl”, “alkyl-aryl”, “arylalkyl”, “alkyl-heteroaryl” and “heteroarylalkyl” mean that alkyl, cycloalkyl, heterocyclyl, aryl and heteroaryl have the meanings defined above and the cycloalkyl, heterocyclyl, aryl and heteroaryl radical is attached via an alkyl radical, preferably C₁-C₈-alkyl radical, partically preferably C₁-C₄-alkyl radical to the compounds of the formulae (I) and (II).

In connection with “alkyl”, “alkenyl” and “alkynyl”, the term “substituted” is, in the context of the present invention, to be understood as meaning the substitution of a hydrogen radical by F, Cl, Br, I, CN, NH₂, NH-alkyl, NH-cycloalkyl, NH-Aryl, NH-heteroaryl, NH-aryl-alkyl, NH-heteroaryl-alkyl, NH-heterocyclyl, NH-alkyl-OH, N(alkyl)₂, N(aryl-alkyl)₂, N(heteroaryl-alkyl)₂, N(heterocyclyl)₂, N(alkyl-OH)₂, NO, NO₂, SH, S-alkyl, S-cycloalkyl, S-aryl, S-heteroaryl, S-aryl-alkyl, S-heteroaryl-alkyl, S-heterocyclyl, S-alkyl-OH, S-alkyl-SH, S-alkyl, S—S-cycloalkyl, S—S-aryl, S—S-heteroaryl, S—S-aryl-alkyl, S—S-heteroaryl-alkyl, S—S-heterocyclyl, SS-alkyl-OH, S—S-alkyl-SH, S—S-alkyl-C(O)—NH-heterocyclyl, OH, O-alkyl, O-cycloalkyl, O-cycloalkyl-alkyl, O-aryl, O-heteroaryl, O-aryl-alkyl, O-heteroaryl-alkyl, O-heterocyclyl, O-heterocyclyl-alkyl, O-alkyl-OH, O-alkyl-O-alkyl, O—SO₂—N(alkyl)₂, O—SO₂—OH, O—SO₂—O-alkyl, O—SO₂—O-cycloalkyl, O—SO₂—O-heterocyclylalkyl, O—SO₂—O-cycloalkyl-alkyl, O—SO₂—O-alkylheterocyclylalkyl, O—SO₂—O-aryl, O—SO₂—O-heteroaryl, O—SO₂—O-arylalkyl, O—SO₂—O-heteroaryl-alkyl, O—SO₂-alkyl, O—SO₂-cycloalkyl, O—SO₂-heterocyclylalkyl, O—SO₂-cycloalkyl-alkyl, O—SO₂-alkylheterocyclylalkyl, O—SO₂-aryl, O—SO₂-heteroaryl, O—SO₂-aryl-alkyl, O—SO₂-heteroaryl-alkyl, O—C(O)-alkyl, O—C(O)-cycloalkyl, O—C(O)-heterocyclylalkyl, O—C(O)-cycloalkyl-alkyl, O—C(O)-alkylheterocyclylalkyl, O—C(O)-aryl, O—C(O)-heteroaryl, O—C(O)-aryl-alkyl, O—C(O)-heteroaryl-alkyl, O—C(O)O-alkyl, O—C(O)O-cycloalkyl, O—C(O)O-heterocyclylalkyl, O—C(O)O-cycloalkyl-alkyl, O—C(O)O-alkylheterocyclylalkyl, O—C(O)O-aryl, O—C(O)O-heteroaryl, O—C(O)O-aryl-alkyl, O—C(O)O-heteroaryl-alkyl, O—C(O)NH-alkyl, O—C(O)NH-cycloalkyl, O—C(O)NH-heterocyclylalkyl, O—C(O)NH-cycloalkyl-alkyl, O—C(O)NH-alkylheterocyclylalkyl, O—C(O)NH-aryl, O—C(O)NH-heteroaryl, O—C(O)NH-aryl-alkyl, O—C(O)NH-heteroaryl-alkyl, O—C(O)N(alkyl)₂, O—C(O)N(cycloalkyl)₂, O—C(O)N(heterocyclylalkyl)₂, O—C(O)N(cycloalkyl-alkyl)₂, O—C(O)N(alkylheterocyclylalkyl)₂, O—C(O)N(aryl)₂, O—C(O)N(heteroaryl)₂, O—C(O)N(aryl-alkyl)₂, O—C(O)N(heteroaryl-alkyl)₂, O—P(O)(OH)₂, O—P(O)(O-metal)₂, O—P(O)(O-alkyl)₂, O—P(O)(O-cycloalkyl)₂, O—P(O)(O-aryl)₂, O—P(O)(O-heteroaryl)₂, O—P(O)(O-aryl-alkyl)₂, O—P(O)(O-heteroaryl-alkyl)₂, O—P(O)(N-alkyl)₂(N-alkyl)₂, O—P(O)(N-cycloalkyl)₂(N-cycloalkyl)₂, O—P(O)(N-heterocyclylalkyl)₂(N-heterocyclyl-alkyl)₂, O—P(O)(N-aryl)₂(N-aryl)₂, O—P(O)(N-heteroaryl)₂(N-heteroaryl)₂, O—P(O)(N-aryl-alkyl)₂(N-aryl-alkyl)₂, O—P(O)(N-heteroaryl-alkyl)₂(N-heteroaryl-alkyl)₂, CHO, C(O)-alkyl, C(S)-alkyl, C(O)-aryl, C(S)-aryl, C(O)-aryl-alkyl, C(S)-aryl-alkyl, C(O)-heterocyclyl, C(O)-heteroaryl, C(O)-heteroaryl-alkyl, C(S)-heterocyclyl, CO₂H, CO₂-alkyl, CO₂-cyclyl, CO₂-heterocyclyl, CO₂-aryl, CO₂-heteroaryl, CO₂-aryl-alkyl, C(O)—NH₂, C(O)NH-alkyl, C(O)NH-aryl, C(O)NH-heterocyclyl, C(O)NH-alkyl-heterocyclyl, C(O)N(alkyl)₂, C(O)N(aryl-alkyl)₂, C(O)N(heteroaryl-alkyl)₂, C(O)N(heterocyclyl)₂, SO-alkyl, SO₂-alkyl, SO₂-aryl, SO₂-aryl-alkyl, SO₂-heteroaryl, SO₂-heteroaryl-alkyl, SO₂NH₂, SO₃H, CF₃, CHO, CHS, alkyl, cycloalkyl, cycloalkyl-alkyl, aryl, aryl-alkyl, heteroaryl, heteroaryl-alkyl, heterocyclyl and/or heterocyclyl-alkyl, where polysubstituted radicals are to be understood as meaning radicals which are polysubstituted, for example di- or trisubstituted, either on different or on the same atoms, for example trisubstituted on the same carbon atom, as in the case of CF₃, —CH₂CF₃, or on different sites, such as in the case of —CH(OH)—CH═CH—CHCl₂. The polysubstitution may be with identical or different substituents.

In relation to “aryl”, “aryl-alkyl”, “alkyl-aryl”, “heteroaryl”, “heteroaryl-alkyl”, “alkyl-heteroaryl”, “heterocyclyl”, “heterocyclyl-alkyl”, “alkyl-heterocyclyl”, “cycloalkyl”, “alkyl-cycloalkyl” and “cycloalkyl-alkyl”, the term “substituted” is, in the context of the present invention, to be understood as meaning mono- or polysubstitution, for example di-, tri- or tetrasubstitution, of one or more hydrogen atoms of the ring system by F, Cl, Br, I, CN, NH₂, NH-alkyl, NH-aryl, NH-heteroaryl, NH-aryl-alkyl, NH-heteroaryl-alkyl, NH-heterocyclyl, NH-alkyl-OH, N(alkyl)₂, NC(O)alkyl, N(aryl-alkyl)₂, N(heteroaryl-alkyl)₂, N(heterocyclyl)₂, N(alkyl-OH)₂, NO, NO₂, SH, S-alkyl, S-aryl, S-heteroaryl, S-aryl-alkyl, S-heteroaryl-alkyl, S-heterocyclyl, S-alkyl-OH, S-alkyl-SH, OH, O-alkyl, O-cycloalkyl, O-cycloalkyl-alkyl, O-aryl, O-heteroaryl, O-aryl-alkyl, O-heteroaryl-alkyl, O-heterocyclyl, O-heterocyclyl-alkyl, O-alkyl-OH, O-alkyl-O-alkyl, O—SO₂—N(alkyl)₂, O—SO₂—OH, O—SO₂—O-alkyl, O—SO₂—O-cycloalkyl, O—SO₂—O-heterocyclylalkyl, O—SO₂—O-cycloalkyl-alkyl, O—SO₂—O-alkylheterocyclylalkyl, O—SO₂—O-aryl, O—SO₂—O-heteroaryl, O—SO₂—O-aryl-alkyl, O—SO₂—O-heteroaryl-alkyl, O—SO₂-alkyl, O—SO₂-cycloalkyl, O—SO₂-heterocyclylalkyl, O—SO₂-cycloalkyl-alkyl, O—SO₂-alkylheterocyclylalkyl, O—SO₂-aryl, O—SO₂-heteroaryl, O—SO₂-aryl-alkyl, O—SO₂-heteroaryl-alkyl, O—C(O)-alkyl, O—C(O)-cycloalkyl, O—C(O)-heterocyclylalkyl, O—C(O)-cycloalkyl-alkyl, O—C(O)-alkylheterocyclylalkyl, O—C(O)-aryl, O—C(O)-heteroaryl, O—C(O)-aryl-alkyl, O—C(O)-heteroaryl-alkyl, O—C(O)O-alkyl, O—C(O)O-cycloalkyl, O—C(O)O-heterocyclylalkyl, O—C(O)O-cycloalkyl-alkyl, O—C(O)O-alkylheterocyclylalkyl, O—C(O)O-aryl, O—C(O)O-heteroaryl, O—C(O)O-aryl-alkyl, O—C(O)O-heteroaryl-alkyl, O—C(O)NH-alkyl, O—C(O)NH-cycloalkyl, O—C(O)NH-heterocyclylalkyl, O—C(O)NH-cycloalkyl-alkyl, O—C(O)NH-alkylheterocyclylalkyl, O—C(O)NH-aryl, O—C(O)NH-heteroaryl, O—C(O)NH-aryl-alkyl, O—C(O)NH-heteroaryl-alkyl, O—C(O)N(alkyl)₂, O—C(O)N(cycloalkyl)₂, O—C(O)N(heterocyclylalkyl)₂, O—C(O)N(cycloalkyl-alkyl)₂, O—C(O)N(alkylheterocyclylalkyl)₂, O—C(O)N(aryl)₂, O—C(O)N(heteroaryl)₂, O—C(O)N(aryl-alkyl)₂, O—C(O)N(heteroaryl-alkyl)₂, O—P(O)(OH)₂, O—P(O)(O-metal)₂, O—P(O)(O-alkyl)₂, O—P(O)(O-cycloalkyl)₂, O—P(O)(O-aryl)₂, O—P(O)(O-heteroaryl)₂, O—P(O)(O-aryl-alkyl)₂, O—P(O)(O-heteroaryl-alkyl)₂, O—P(O)(N-alkyl)₂(N-alkyl)₂, O—P(O)(N-cycloalkyl)₂(N-cycloalkyl)₂, O—P(O)(N-heterocyclylalkyl)₂(N-hetero-cycloalkyl)₂, O—P(O)(N-aryl)₂(N-aryl)₂, O—P(O)(N-heteroaryl)₂(N-heteroaryl)₂, O—P(O)(N-aryl-alkyl)₂(N-aryl-alkyl)₂, O—P(O)(N-heteroaryl-alkyl)₂(N-heteroaryl-alkyl)₂, CHO, C(O)-alkyl, C(S)-alkyl, C(O)-aryl, C(S)-aryl, C(O)-aryl-alkyl, C(S)-aryl-alkyl, C(O)-heterocyclyl, C(S)-heterocyclyl, CO₂H, CO₂-alkyl, CO₂-aryl-alkyl, C(O)—NH₂, C(O)NH-alkyl, C(O)NH-aryl, C(O)NH-heterocyclyl, C(O)N(alkyl)₂, C(O)N(aryl-alkyl)₂, C(O)N(heteroaryl-alkyl)₂, C(O)N(heterocyclyl)₂, SO-alkyl, SO₂-alkyl, SO₂-aryl, SO₂-aryl-alkyl, SO₂-heteroaryl, SO₂-heteroaryl-alkyl, SO₂NH₂, SO₃H, CF₃, CHO, CHS, alkyl, cycloalkyl, cycloalkyl-alkyl, aryl, aryl-alkyl, heteroaryl, heteroaryl-alkyl, heterocyclyl and/or heterocyclyl-alkyl, where polysubstituted radicals are to be understood as meaning radicals which are polysubstituted, for example di- or trisubstituted, either on different or on the same atoms, for example trisubstituted on the same carbon atom, as in the case of CF₃, —CH₂CF₃, or on different sites, such as in the case of —CH(OH)—CH═CH—CHCl₂. The polysubstitution may be with identical or different substituents.

In the context of the present invention, the expressions “mono-alkylamino” and “di-alkylamino” are to be understood as meaning that one and two, respectively, alkyl radicals according to the above definition are attached to a nitrogen atom. The attachment to the compounds of the formula (I) is via the nitrogen atom. Examples are ethylamino, dimethylamino and isopropylethylamino.

In the context of the present invention, the expression “cyano-alkyl” is to be understood as meaning that an alkyl radical according to the above definition is attached to a cyano group. The attachment to the compounds of the formulae (I) and (II) is via the cyano group. Examples are methylcyano and n-propylcyano.

In the context of the present invention, the expression “carbonyl” is to be understood as meaning that an alkyl, cycloalkyl, cyloalkyl-alkyl, alkyl-cycloalkyl, aryl, heteroaryl, aryl-alkyl, alkyl-aryl, heteroaryl-alkyl, alkyl-heteroaryl, heterocyclyl, alkyl-heterocyclyl and/or heterocyclyl-alkyl radical according to the above definition is attached to a —C(O) group. The attachment to the compounds of the formulae (I) and (II) is via the —C(O)— group. Examples are —C(O)CH₃, —C(O)CH₂CH₃, —C(O)-isopropyl and —C(O)-tBu (tBu=tert-butyl).

In the context of the present invention, the expression “carboxyl” is to be understood as meaning that an alkyl, cycloalkyl, cyloalkyl-alkyl, alkyl-cycloalkyl, aryl, heteroaryl, aryl-alkyl, alkyl-aryl, heteroaryl-alkyl, alkyl-heteroaryl, heterocyclyl, alkyl-heterocyclyl and/or heterocyclyl-alkyl radical according to the above definition is attached to a —C(O)O— group. The attachment to the compounds of the formulae (I) and (II) is via the —C(O)O— group. Examples are —C(O)O—CH₃ and —C(O)O-phenyl.

In the context of the present invention, the expression “carboxy-alkyl” is to be understood as meaning that a carboxyl group according to the above definition is attached to an alkyl radical according to the above definition. The attachment to the compounds of the formulae (I) and (II) is via the alkyl radical.

In the context of the present invention, the expression “alkoxy” is to be understood as meaning that an alkyl radical according to the above definition is attached to an oxygen atom. The attachment to the compounds of the formulae (I) and (II) is via the oxygen atom. Examples are methoxy, ethoxy and n-propyloxy.

In the terms “alkoxycarbonyl”, “aryl-alkoxy”, “heteroaryl-alkoxy”, “alkoxycarbonylamino”, “alkoxycarbonylamino-alkyl”, the expression “alkoxy” has the meaning defined above. In the case of “aryl-alkoxy” and “heteroaryl-alkoxy”, the attachment to the compounds of the formulae (I) and (II) is via the oxygen atom. Examples are benzyloxy and indolyloxy.

In the case of “alkoxycarbonyl”, the attachment to the compounds of the formulae (I) and (II) is via the —C(O) group.

In the case of “alkoxycarbonyl-alkyl”, the attachment to the compounds of the formulae (I) and (II) is via the alkyl radical according to the meaning defined above.

In the case of “alkoxycarbonylamino”, the attachment to the compounds of the formulae (I) and (II) is via the amino group; in the case of “alkoxycarbonylamino-alkyl” via the alkyl radical according to the meaning defined above.

In this context of the present invention, the expression “metal” includes metal ions such as sodium, potassium, lithium, magnesium, calcium, zinc and manganese ions.

In the context of the present invention, the expression “halogen” includes the halogen atoms fluorine, chlorine, bromine and iodine.

If the compounds according to the invention have at least one centre of asymmetry, they can be present in the form of their racemates, in the form of the pure enantiomers and/or diastereomers or in the form of mixtures of these enantiomers and/or diastereomers, both as such and as pharmaceutically acceptable salts of these compounds. The mixtures can be present in any desired mixing ratio of the stereoisomers.

Thus, for example, the compounds according to the invention which have one or more centres of chirality and which occur as racemates can be separated into their optical isomers, that is enantiomers or diastereomers, by methods known per se. The separation can be carried out by column separation on chiral phases or by recrystallization from an optically active solvent or using an optically active acid or base or by derivatization with an optically active reagent, such as, for example, an optically active alcohol, and subsequent removal of the radical.

The compounds according to the invention can be present in the form of their double-bond isomers as “pure” E or Z isomers or in the form of mixtures of these double-bond isomers.

If possible, the compounds according to the invention can be present in the form of tautomers.

The compounds according to the invention can, if they have a sufficiently basic group, such as, for example, a primary, secondary or tertiary amine, be converted into their physiologically acceptable salts using inorganic and organic acids. Preferably, the pharmaceutically acceptable salts of the compounds according to the invention are formed with hydrochloric acid, hydrobromic acid, sulphuric acid, phosphoric acid, methanesulphonic acid, p-toluenesulphonic acid, carbonic acid, formic acid, acetic acid, trifluoroacetic acid, sulphoacetic acid, oxalic acid, malonic acid, maleic acid, succinic acid, tartaric acid, racemic acid, malic acid, embonic acid, mandelic acid, fumaric acid, lactic acid, citric acid, glutamic acid or aspartic acid. The salts formed are, inter alia, hydrochlorides, hydrobromides, sulphates, hydrogensulphates, phosphates, methanesulphonates, tosylates, carbonates, bicarbonates, formates, acetates, triflates, sulphoacetates, oxalates, malonates, maleates, succinates, tartrates, malates, embonates, mandelates, fumarates, lactates, citrates, glutamates and aspartates. The stoichiometry of the salts formed of the compounds according to the invention can in this case be an integral or nonintegral multiple of one.

The compounds according to the invention can, if they contain a sufficiently acidic group, such as, for example, the carboxyl group or the phosphoric acid group, be converted into their physiologically acceptable salts with inorganic and organic bases. Suitable inorganic bases are, for example, sodium hydroxide, potassium hydroxide, calcium hydroxide, as organic bases ethanolamine, diethanolamine, triethanolamine, cyclohexylamine, dibenzylethylenediamine and lysine. The stoichiometry of the salts formed of the compounds according to the invention can in this case be an integral or nonintegral multiple of one.

The compounds of the formulae (I) and (II) according to the invention can, if they contain groups capable of forming coordination compounds, be converted into coordination compounds/complex compounds using transition metals. Preference is given to forming the pharmaceutically acceptable coordination compounds/complex compounds of the compounds according to the formulae (I) and (II) according to the invention with platinum salts.

The stoichiometry of the coordination compounds/complex compounds formed of the compounds according to the invention can in this case be an integral or nonintegral ratio of the compounds according to the invention as ligands for the transition metal.

Preference is also given to solvates and in particular hydrates of the compounds according to the invention which can be obtained, for example, by crystallization from a solvent or from aqueous solution. Here, one, two, three or any number of solvate or water molecules may bind to the compounds according to the invention forming solvates and hydrates.

It is known that chemical substances form solids which are present in various atomic states, which are described as polymorphic forms or modifications. The different modifications of a polymorphic substance can differ greatly in their physical properties.

The compounds according to the invention can be present in various polymorphic forms, where certain modifications may be metastable.

The compounds according to the invention may also be present in the form of any prodrugs, such as, for example, esters, carbonates, carbamates, ureas, amides or phosphates, in which the form that is actually biologically active is only released by metabolization.

It is known that chemical substances are converted in the body to metabolites which, if appropriate, may also cause the desired biological effect—in certain cases even in a more pronounced form.

Such prodrugs and metabolites of compounds according to the invention are to be considered as being part of the invention.

Surprisingly, using the compounds according to the invention, it has now been possible to demonstrate antiproliferative activity in various cellular tumor models.

Furthermore, it was possible to show that the compounds according to the invention are potent inhibitors of tubulin polymerization and/or inhibitors of microtubuli-based motor proteins, for example kinesins and dyneins, in particular mitotic kinesins. Kinesins have been characterized and classified in detail in Miki H et al. (Proc. Natl. Acad. Sci. USA 2001, 98: 7004-7011) and include, for example, kinesin superfamily (KIF) protein, N-1 kinesins, KIF5A, KIF5B, KIF5C, N-2 kinesins, Eg5 (KIF11, KSP), BimC (KIF8), N-3 kinesins, KIF1A, KIF1B, KIF1C, KIF13A, KIF13B, KIF14, KIF16A, KIF16B, N-4 kinesins, KIF3A, KIF3B, KIF3C, KIF17, N-5 kinesins, KIF4, KIF4A, KIF4B, KIF21A, KIF21B, N-6 kinesins, KIF23 (MKLP1, CHO1), KIF20A (Rab6-KIF), KIF20B (KIpMPP1), MKLP2, N-7 kinesins, KIF10 (CENP-E), N-8 kinesins, KIF18A, KIF18B, KIF22 (Kid), KIF19A, KIF19B, N-9 kinesins, KIF12, N-10 kinesins, KIF15 (HkIp2), N-11 kinesins, KIF24, KIF25 (KNSL3), KIF26A, KIF26B, M-kinesins, KIF2A (KIF2), KIF2B, KIF2C (MCAK), C-1 kinesins, KIFC1, C-2 kinesins, KIFC2, KIFC3, KIF6, KIF7 and KIF9.

By virtue of the surprising dual mechanism of action, the compounds according to the invention are particularly suitable as medicaments for the treatment of benign and malignant oncoses in humans and animals. Both the tubulin skeleton and the motor proteins play an important role in mitosis, a specific phase of the cell cycle. By the advantageous inhibition of one or both targets, the correct formation of the mitotic spindle and thus the distribution of the genetic material to the daughter cells is prevented. The cell cycle of the tumor cell undergoing uncontrolled division is arrested. As a result, this leads to the tumor cell dying by programmed cell death (apoptosis).

In this manner, an effective treatment of the tumor is possible, and using the compounds according to the invention, it is possible to overcome (multiple) resistances against known tumor therapeutics.

Furthermore, by virtue of their specificity and/or the dual mechanism of action, the compounds according to the invention advantageously have no serious side effects, such as, for example, neurotoxicity.

According to a further aspect, the object of the invention was surprisingly achieved by providing a process for preparing the compounds according to the invention.

In the context of the present invention, the compounds according to the invention can be administered to all known mammals, in particular man, for treatment and/or prophylaxis.

In another preferred embodiment, the compounds according to the invention are provided for the applications mentioned above, the mammal being selected from the group consisting of: “man, useful animal, livestock, pet, cattle, cow, sheep, pig, goat, horse, pony, donkey, hinny, mule, hare, rabbit, cat, dog, guinea pig, hamster, rat, mouse” and is preferably man.

According to a further aspect, the object of the invention was surprisingly achieved by providing anthracene compounds according to the formula (II) 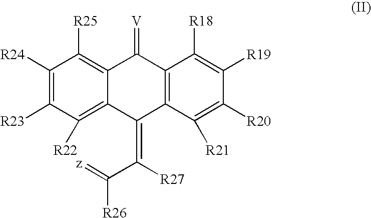
in which:
substituent V is independently selected from the group consisting of: “O, S, NR28, N—OR29, geminally attached hydrogen and hydroxyl”;
substituent Z is independently selected from the group consisting of: “O, S, NR30, N—OR31”;
substituents R18, R19, R20, R21, R22, R23, R24, R25 independently of one another are independently selected from the group consisting of: “hydrogen, unsubstituted or substituted alkyl, unsubstituted or substituted cycloalkyl, unsubstituted or substituted cycloalkyl-alkyl, unsubstituted or substituted heterocyclyl, unsubstituted or substituted heterocyclyl-alkyl, unsubstituted or substituted aryl, unsubstituted or substituted aryl-alkyl, unsubstituted or substituted heteroaryl, unsubstituted or substituted heteroaryl-alkyl, amino, mono-alkylamino, di-alkylamino, halogen, —F, —Cl, —Br, —I, alkyl substituted by one or more fluorine atoms, trifluoromethyl, cyano, straight-chain or branched cyano-alkyl, carbonyl, carboxyl, —COOH, alkoxycarbonyl, carboxy-alkyl, alkoxycarbonyl-alkyl, hydroxyl, alkoxy, aryl-alkoxy, benzyloxy, heteroaryl-alkoxy, alkoxycarbonylamino, alkoxycarbonylamino-alkyl”;
substituent R26 is independently selected from the group consisting of: “unsubstituted or substituted alkyl, unsubstituted or substituted cycloalkyl, unsubstituted or substituted cycloalkyl-alkyl, unsubstituted or substituted heterocyclyl, unsubstituted or substituted heterocyclyl-alkyl, unsubstituted or substituted aryl, unsubstituted or substituted aryl-alkyl, unsubstituted or substituted heteroaryl, unsubstituted or substituted heteroaryl-alkyl, —OR32, —NR33R34”;
substituent R27 is independently selected from the group consisting of: “hydrogen, unsubstituted or substituted alkyl, unsubstituted or substituted aryl, unsubstituted or substituted aryl-alkyl, unsubstituted or substituted heteroaryl, unsubstituted or substituted heteroaryl-alkyl”;
substituents R28, R29, R30, R31, R32, R33, R34 independently of one another are independently selected from the group consisting of: “hydrogen, unsubstituted or substituted alkyl, unsubstituted or substituted cycloalkyl, unsubstituted or substituted cycloalkyl-alkyl, unsubstituted or substituted heterocyclyl, unsubstituted or substituted heterocyclyl-alkyl, unsubstituted or substituted aryl, unsubstituted or substituted aryl-alkyl, unsubstituted or substituted heteroaryl, unsubstituted or substituted heteroaryl-alkyl”;
which can be used for preparing a medicament.

Medicaments comprising at least one anthracene compound according to the formula (II) are likewise comprised in the context of the present invention.

The substitution “geminally attached hydrogen and hydroxyl” in conjunction with the substituent V is to be understood in the context of the present invention as meaning that both “H” and “OH” are attached to one and the same carbon atom.

In a preferred embodiment, anthracene compounds according to the formula (II) as shown above are provided in which:
substituent V is independently selected from the group consisting of: “O, S, NH, geminally attached hydrogen and hydroxyl”;
substituent Z is independently “O or S”;
substituents R18, R19, R20, R21, R22, R23, R24, R25 are independently “hydrogen, fluorine, chlorine, bromine, iodine, hydroxyl, amino, nitro, cyano, methyl, trifluoromethyl”;
substituent R26 is independently selected from the group consisting of: “unsubstituted or substituted heterocyclyl, unsubstituted or substituted aryl, unsubstituted or substituted heteroaryl, —OR32, —NR33R34”;
substituent R27 is independently “hydrogen, alkyl”;
which can be used for preparing a medicament.

Medicaments comprising at least one anthracene compound according to the formula (II) and this preferred embodiment are likewise comprised in the context of the present invention.

In a preferred embodiment, anthracene compounds according to the formula (II) as shown above are provided in which:
substituent R27 is independently selected from the group consisting of: “3,4-dimethoxy-phenyl; 3,4-dihydroxy-phenyl; 4-methoxy-thiophen-2-yl, thiomorpholin-4-yl; 4-methoxy-phenyl; 4-(4-methoxy-phenyl)-piperazin-1-yl, 2-hydroxy-3,4-dimethoxy-phenyl, 4-methylphenyl; 4-hydroxyphenyl; thiophen-2-yl; 3-hydroxyphenyl; 3-methoxy-phenyl; 2,6-dimethoxy-phenyl, 3,4,5-trimethoxy-phenyl, hydroxyl; methoxy; phenyl; 4-chlorophenyl; 4-bromophenyl; 4-fluorophenyl; 3-chlorophenyl; 3-bromophenyl; 3-fluorophenyl; 4-fluoro-3-hydroxy-phenyl”;
which can be used for preparing a medicament.

Medicaments comprising at least one anthracene compound according to the formula (II) and this preferred embodiment are likewise comprised in the context of the present invention.

A preferred embodiment provides anthracene compound compounds 1 to 43 for preparing a medicament.

Medicaments comprising at least one anthracene compound compound 1 to 43 are likewise comprised in the context of the present invention.

In the context of the present invention, the compounds according to the invention can be used for the treatment and/or prophylaxis of physiological and/or pathophysiological conditions which can be treated by inhibiting tubulin polymerization and/or by inhibiting microtubuli-based motor proteins.

In a preferred embodiment, the microtubuli-based motor proteins are selected from the group consisting of: “kinesins, dyneins, kinesin superfamily (KIF) protein, N-1 kinesins, KIF5A, KIF5B, KIF5C, N-2 kinesins, Eg5 (KIF11, KSP), BimC (KIF8), N-3 kinesins, KIF1A, KIF1B, KIF1C, KIF13A, KIF13B, KIF14, KIF16A, KIF16B, N-4 kinesins, KIF3A, KIF3B, KIF3C, KIF17, N-5 kinesins, KIF4, KIF4A, KIF4B, KIF21A, KIF21B, N-6 kinesins, KIF23 (MKLP1, CHO1), KIF20A (Rab6-KIF), KIF20B (KIpMPP1), MKLP2, N-7 kinesins, KIF10 (CENP-E), N-8 kinesins, KIF18A, KIF18B, KIF22 (Kid), KIF19A, KIF19B, N-9 kinesins, KIF12, N-10 kinesins, KIF15 (HkIp2), N-11 kinesins, KIF24, KIF25 (KNSL3), KIF26A, KIF26B, M-kinesins, KIF2A (KIF2), KIF2B, KIF2C (MCAK), C-1 kinesins, KIFC1, C-2 kinesins, KIFC2, KIFC3, KIF6, KIF7, KIF9” and preferably selected from the group consisting of: “Eg5 (KIF11, KSP), KIF4, KIF4A, KIF4B, KIF10 (CENP-E), KIF23 (MKLP1, CHO1)”.

In a further preferred embodiment, the compounds according to the invention can be used for the treatment or prophylaxis of malignant tumors, benign tumors, solid tumors, sarcomas, carcinomas, hyperproliferative disorders, carcinoids, Ewing sarcomas, Kaposi sarcomas, brain tumors, tumors originating from the brain and/or the nervous system and/or the meninges (WO 99/01764), gliomas, neuroblastomas, stomach cancer, kidney cancer, kidney cell carcinomas, prostate cancer, prostate carcinomas, connective tissue tumors, soft tissue sarcomas, pancreas tumors, liver tumors, head tumors, neck tumors, oesophageal cancer, thyroid cancer, osteosarcomas, retinoblastomas, thymoma, testicular cancer, lung cancer, bronchial carcinomas, breast cancer, mamma carcinomas, intestinal cancer, colorectal tumors, colon carcinomas, rectum carcinomas, gynaecological tumors, ovary tumors/ovarian tumors, uterine cancer, cervical cancer, cervix carcinomas, cancer of body of uterus, corpus carcinomas, endometrial carcinomas, urinary bladder cancer, bladder cancer, skin cancer, basaliomas, spinaliomas, melanomas, intraocular melanomas, leukaemias, chronic leukaemias, acute leukaemias and/or lymphomas. These indications are also physiological and/or pathophysiological conditions which can be treated by inhibiting tubulin polymerization and/or by inhibiting microtubuli-based motor proteins.

According to a further aspect of the present invention, the object of the invention was surprisingly achieved by providing the compounds according to the invention according to the aspects, preferred embodiments and applications shown above, for use for preparing a medicament, the medicament additionally comprising at least one further pharmacologically active substance.

According to a further aspect of the present invention, the object of the invention was surprisingly achieved by providing the compounds according to the invention according to the aspects, preferred embodiments and applications shown above, for use for preparing a medicament, the medicament being administered before and/or during and/or after the treatment with at least one further pharmacologically active substance.

According to a further aspect of the present invention, the object of the invention was surprisingly achieved by providing the compounds according to the invention according to the aspects, preferred embodiments and applications shown above, for use for preparing a medicament, the medicament being administered before and/or during and/or after treatment with radiotherapy and/or surgery.

Here, in the context of the present invention, the compounds according to the invention can be administered as individual substances or in combination with any known pharmacologically active substances in a combination therapy as shown.

In a preferred embodiment, the compounds according to the invention are provided for the applications shown above, the further pharmacologically active substance being selected from the group consisting of: “DNA topoisomerase I and/or II inhibitors, DNA intercalators, alkylating agents, microtubuli destabilizers, hormone and/or growth factor receptor agonists and/or antagonists, inhibitors of signal transduction, antibodies against growth factors and their receptors, kinase inhibitors, antimetabolites”.

In a preferred embodiment, the compounds according to the invention are provided for the applications shown above, the further pharmacologically active substance being selected from the group consisting of: “actinomycin D, aminoglutethimide, asparaginase, avastin, azathioprine, BCNU (carmustine), bleomycin, busulfan, carboplatin, CCNU (lomustine), chlorambucil, cisplatin, colaspase, cyclophosphamide, cytarabine, dactinomycin, daunorubicin, diethylstilbestrol, doxorubicin (adriamycin), DTIC (dacarbacin), epirubicin, erbitux, erythrohydroxynonyladenine, ethynyloestradiol, etoposide, fludarabine phosphate, fluoxymesterone, flutamide, gemcitabine, Gleevec/Glivec, Herceptin, hexamethylmelamine, hydroxyurea, hydroxyprogesterone caproate, idarubicin, ifosfamide, interferon, iressa, irinotecan, L-asparaginase, leucovorin, mechlorethamine, medroxyprogesterone acetate, megestrol acetate, melphalan, mesna, methotrexate, mitomycin C, mitotane, mitoxantrone, N-phosphonoacetyl-L-aspartate (PALA), oxaliplatin, pentostatin, plicamycin, prednisolone, prednisone, procarbazine, raloxifen, rapamycin, semustine, sorafenib, streptozocin, tamoxifen, tarceva, taxotere, teniposide, testosterone propionate, thioguanine, thiotepa, topotecan, trimethylmelamine, uridine, vinblastine, vincristine, vindesine, vinorelbine, 2′,2′-difluorodeoxycytidine, 5-fluorodeoxyuridine monophos-phate, 5-azacytidine cladribine, 5-fluorodeoxyuridine, 5-fluorouarcil (5-FU), 6-mercaptopurine”.

The medicaments according to the invention can be administered as liquid, semisolid and solid medicinal forms. This takes place in the manner suitable in each case in the form of aerosols, powders, dusting powders and epipastics, tablets including coated tablets, emulsions, foams, solutions, suspensions, gels, ointments, pastes, pills, pastilles, capsules or suppositories.

The medicaments according to the invention can be administered in a suitable dosage form to the skin, epicutaneously as solution, suspension, emulsion, foam, ointment, paste or plaster; via the oral and lingual mucosa, buccally, lingually or sublingually as tablet, pastille, coated tablet, linctus or gargle; via the gastric and intestinal mucosa, enterally as tablet, coated tablet, capsule, solution, suspension or emulsion; via the rectal mucosa, rectally as suppository, rectal capsule or ointment; via the nasal mucosa, nasally as drops, ointments or spray; via the bronchial and alveolar epithelium, by the pulmonary route or by inhalation as aerosol or inhalant; via the conjunctiva, conjunctivally as eyedrops, eye ointment, eye tablets, lamellae or eye lotion; via the mucosa of the genital organs, intravaginally as vaginal suppositories, ointments and douche, by the intrauterine route as uterine pessary; via the urinary tract, intraurethrally as irrigation, ointment or bougie; into an artery, intraarterially as injection; into a vein, intravenously as injection or infusion; into the skin, intracutaneously as injection or implant; under the skin, subcutaneously as injection or implant; into the muscle, intramuscularly as injection or implant; into the abdominal cavity, intraperitoneally as injection or infusion.

Oral administration can take place for example in solid form as tablet, capsule, gel capsule, coated tablet, granules or powder, but also in the form of a drinkable solution. The novel compounds according to the invention as defined above can for oral administration be combined with known and ordinarily used, physiologically acceptable auxiliaries and carriers, such as, for example, gum arabic, talc, starch, sugars such as, for example, mannitol, methylcellulose, lactose, gelatin, surface-active agents, magnesium stearate, cyclodextrins, aqueous or nonaqueous carriers, diluents, dispersants, emulsifiers, lubricants, preservatives and flavourings (for example essential oils). The compounds according to the invention can also be dispersed in a microparticulate, for example nanoparticulate, composition.

Non-oral administration can take place, for example, by intravenous, subcutaneous, intramuscular injection of sterile aqueous or oily solutions, suspensions or emulsions, by means of implants or by ointments, creams or suppositories. Administration as sustained-release form is also possible where appropriate. Implants may comprise inert materials, for example biodegradable polymers or synthetic silicones, such as, for example, silicone rubber. Intravaginal administration is possible, for example, by means of vaginal rings. Intrauterine administration is possible, for example, by means of diaphragms or other suitable intrauterine devices. Transdermal administration is additionally provided, in particular by means of a formulation suitable for this purpose and/or suitable means, such as, for example, plasters.

As already explained above, the novel compounds according to the invention can also be combined with other pharmaceutically active compounds. It is possible for the purpose of a combination therapy to administer the individual active ingredients simultaneously or separately, in particular either by the same route (for example orally) or by separate routes (for example orally and as injection). They may be present and administered in identical or different amounts in a unit dose. It is also possible to use a particular dosage regimen when this appears appropriate. It is also possible in this way to combine a plurality of the novel compounds according to the invention with one another.

The dosage may vary within a wide range depending on the type of indication, the severity of the disorder, the mode of administration, the age, gender, body weight and sensitivity of the subject to be treated. It is within the ability of a skilled worker to determine a “pharmacologically effective amount” of the combined pharmaceutical composition. Administration can take place in a single dose or a plurality of separate dosages.

A suitable unit dose is not particularly limited and is, for example, from 0.001 mg to 100 mg of the active compound, i.e. at least one compound according to the invention and, where appropriate, a further pharmaceutically active substance, per kg of a patient's body weight.

A further aspect of the present invention accordingly includes pharmaceutical compositions comprising a pharmacologically active amount of at least one compound according to the invention, preferably compound 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42 and/or compound 43 and, where appropriate, pharmaceutically acceptable carriers and/or auxiliaries, of the present invention.

Preferred and particularly preferred pharmaceutical compositions are those comprising at least one of the preferred compounds according to the invention mentioned above. In pharmaceutical compositions according to the present invention it is possible, in addition to at least one compound according to the invention as defined above, for at least one further pharmaceutically active substance also to be present, as already detailed above.

In the pharmaceutical compositions according to the invention, at least one of the novel compounds according to the invention as defined above is present in a pharmacologically active amount, preferably a unit dose, for example the aforementioned unit dose, preferably in an administration form which makes oral administration possible.

Concerning pharmaceutical compositions comprising compounds according to the invention, and concerning the use of the compounds according to the invention as medicaments, reference may be made to that already said in connection with the application of the novel compounds according to the invention, in relation to possible uses and administrations.

According to a further aspect of the present invention, the object of the invention was surprisingly achieved by providing a kit comprising a pharmacologically active amount of at least one preferred compound according to the invention, as shown above, and a pharmacologically active amount of at least one further pharmacologically active substance as defined above.

Chemical Synthesis:

The compounds of the formulae (I) and (II) can be obtained, for example, according to Scheme 1 below. The radicals R18 to R34, which, in formula (II), differ from formula (I), are to be considered as being analogous to the radicals R1 to R17 of formula (I).

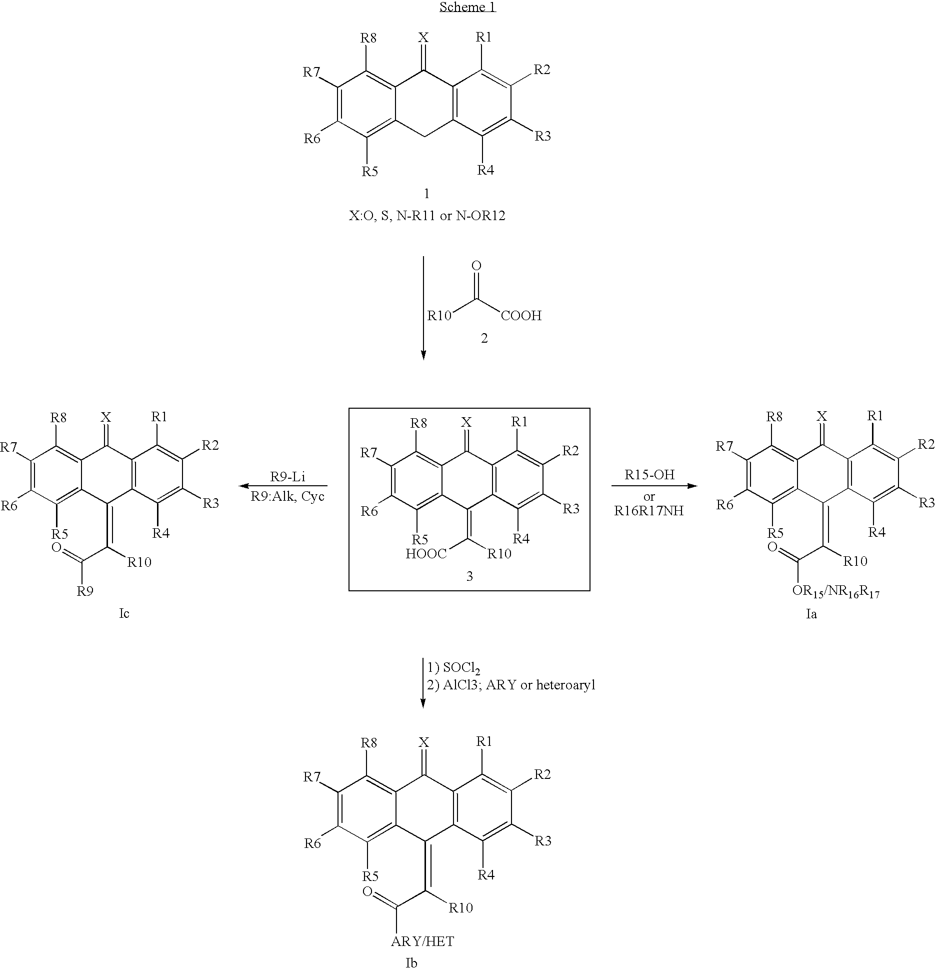

The starting materials 1 and 2 are either commercially available or can be prepared by procedures known per se. Compound 3 is a useful intermediate for preparing the compounds of the formulae (I) and (II) according to the invention.

The solvents and auxiliaries to be used, if appropriate, and the reaction parameters to be applied, such as reaction temperature and reaction time, are familiar to the person skilled in the art owing to his expert knowledge.

The compounds according to the invention, in particular the compounds 1 to 43, were named using the AutoNom 2000 software (ISIS™/Draw 2.5; MDL).

The contents of all patents and references cited are hereby incorporated by way of reference.

The invention is illustrated in more detail by the examples below; however, it is not limited to these examples.

### EXAMPLES

### I) Preparation of Compounds According to the Invention

### Example 1

### 10-[2-(3,4-Dihydroxy-phenyl)-2-oxo-ethylidene]-10H-anthracen-9-one (Compound 1)

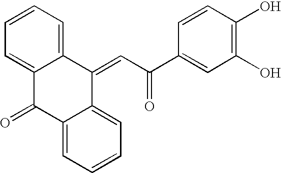

### Step 1: Preparation of 10-oxo-9(10H)-anthracenylideneacetic acid

10.0 g of 9(10H)anthracenone (51.5 mmol) and 9.2 g of glyoxylic acid monohydrate (10.0 mmol) were suspended in 150 ml of ethanol. Under nitrogen, the mixture was heated to the boil. During heating, 6 drops of piperidine were added, whereupon the 9(10H)anthracenone went into solution.

After 4 h, the reaction had ended. After cooling to room temperature, the reaction mixture was then poured into 400 ml of H₂O and 10 ml of 6N HCl and extracted with dichloromethane (5×50 ml). The combined organic phases were repeatedly washed with water, dried over Na₂SO₄ and, under water pump vacuum (rotary evaporator), concentrated to a small volume (30-40 ml).

On standing in an ice bath (1 h), the colourless to slightly yellowish 10-oxo-9(10H)-anthracenylideneacetic acid precipitated out. The product was filtered off with suction and the filter cake was compressed and washed with 100 ml of cold dichloromethane. This resulted in unreacted 9(10H)anthracenone being dissolved. To obtain more acid, the filtrate was reconcentrated and treated as above. The pale yellow powder was dried (drying pistol) and used without further purification for the next step 2.

Yield 7.60 g (59%) pale yellow solid

Melting point m.p.: 180-182° C.

FTIR: 1684,1663 cm⁻¹

1H-NMR (DMSO-d6) δ=8.15-8.07 (m, 2H), 7.93-7.91 (m, 1H), 7.77-7.60 (m, 3H), 7.52-7.43 (m, 2H), 7.08 (s, 1H).

### Step 2: Preparation of 10-oxo-9(10H)-anthracenylideneacetyl chloride

2.0 g of 10-oxo-9(10H)-anthracenylideneacetic acid (7.99 mmol) were suspended in 30 ml of thionyl chloride. 2 drops of N,N-dimethylformamide were added, and, under reflux, the mixture was heated to the boil for 2 h. The mixture was then allowed to cool to room temperature, and the thionyl chloride was removed under water pump vacuum. Twice, in each case 30 ml of n-hexane were added to the residue and in each case removed to dryness under water pump vacuum. The 10-oxo-9(10H)-anthracenylidene-acetyl chloride obtained in this manner could be reacted further, without further purification, in step 3.

Yield 2.0 g (93%) yellow solid

Melting point m.p.: 115° C. (decomp.)

FTIR: 1757,1658 cm⁻¹

1H-NMR (DMSO-d6) δ=8.15-8.07 (m, 3H), 7.93-7.91 (m, 1H), 7.77-7.60 (m, 4H), 7.08 (s, 1H).

### Step 3a: Synthesis of 10-[2-(3,4-dimethoxy-phenyl)-2-oxo-ethylidene]-9(10H)-anthracenone (Compound 6)

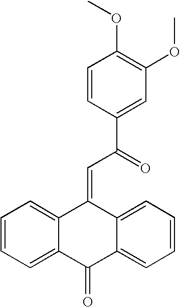 With cooling (ice/sodium chloride bath), 2.15 g (8.0 mmol) of 10-oxo-9(10H)-anthracenylideneacetyl chloride were suspended in 30 ml of 1,2-dichloroethane. 1.07 g of anhydrous AlCl₃ (8 mmol) were then added in one portion. The mixture was stirred in the ice-bath for 10 min, and 1.11 g (8.00 mmol) of 1,2-dimethoxybenzene, dissolved in ml of 1,2-dichloroethane, were then added dropwise. The colour of the mixture turned dark, and the 10-[2-(3,4-dimethoxy-phenyl)-2-oxo-ethylidene]-9(10H)-anthracenone went into solution. The mixture was stirred with ice-cooling (monitored by TLC, SiO₂/DCM). After the reaction had ended (1-2 h), the mixture was poured into 300 ml of water and 50 ml of 6N HCl, stirred for 10 minutes and then extracted with dichloromethane (4×50 ml). The combined organic phases were repeatedly washed with water and dried over sodium sulphate. The solvent was removed under water pump vacuum and the residue was purified by column chromatography on silica gel (EA/PE 7:3), which gave 0.97 g of 10-[2-(3,4-dimethoxy-phenyl)-2-oxo-ethylidene]-9(10H)-anthracenone (compound 6) as a yellow solid.

Yield: 0.97 g (33% of theory) yellow solid

Melting point m.p.: 160° C.

FTIR: 1655,1639 cm⁻¹

¹H-NMR (DMSO-d6) δ=8.32 (d, 1H); 8.20 (d, 1H); 8.14 (d, 1H); 7.81 (t, 1H); 7.70 (s, 1H); 7.69 (t, 1H); 7.60 (d, 1H); 7.55 (t, 1H); 7.46-7.52 (m, 3H); 7.02 (d, 1H); 3.82 (s, 3H); 3.79 (s, 3H) ppm.

### Step 3b: 10-[2-(3,4-Dihydroxy-phenyl)-2-oxo-ethylidene]-10H-anthracen-9-one (Compound 1)

At −70° C. (acetone/dry ice), a solution of 342 mg (1.0 mmol) of 10-[2-(3,4-dimethoxy-phenyl)-2-oxo-ethylidene]-9(10H)-anthracenone (compound 6) in 10 ml of dried dichloromethane was added dropwise to a solution of 5-7 times the molar amount of BBr₃ in 20 ml of DCM (N₂ atmosphere). The mixture was stirred for 24 h, during which time it was allowed to slowly warm to room temperature. The mixture was then poured into excess water and shaken vigorously. The mixture was extracted with ethyl acetate, and the extract was dried over Na₂SO₄ and concentrated under water pump vacuum. The residue was purified by column chromatography on silica gel (EA/PE 7:3). After concentration of the pure fractions, 102 mg of 10-[2-(3,4-dihydroxy-phenyl)-2-oxo-ethylidene]-10H-anthracen-9-one (compound 1) crystallized on addition of hexane.

Yield: 102 mg (30% of theory) yellow, amorphous powder

Melting point m.p.: 217° C.

FTIR: 3255 (br), 1634 cm⁻¹

¹H-NMR (d6-DMSO) δ=8.27 (d, 1H, J=8.21 Hz), 8.19-8.17 (m, 1H), 8.14-8.11 (m, 1H), 7.82-7.78 (m, 1H), 7.68-7.64 (m, 2H), 7.54-7.47 (m, 3H), 7.36-7.33 (m, 2H), 6.78-6.76 (d, 1H);

The following compounds of the formulae (I) and (II) were synthesized analogously to the synthesis route in Scheme 1:

### Example 2

### 10-[2-(4-Methoxy-thiophen-2-yl)-2-oxo-ethylidene]-10H-anthracen-9-one (Compound 2)

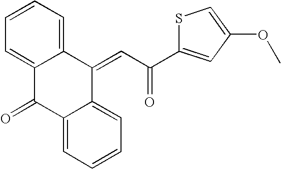

Melting point m.p.: 200-201° C.

FTIR: 1648,1621 cm⁻¹

1H-NMR (DMSO-d6) δ=8.18 (d, 1H); 8.12-8.15 (m, 2H); 8.04 (d, 1H); 7.83 (t, 1H); 7.65-7.70 (m, 2H); 7.52-7.59 (m, 2H); 7.52 (s, 1H); 7.11 (d, 1H); 3.81 (s, 3H) ppm.

### Example 3

### 10-(2-Oxo-2-thiomorpholin-4-ylethylidene)-10H-anthracen-9-one (Compound 3)

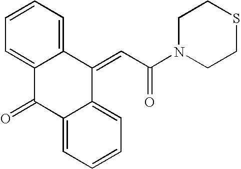

Melting point m.p.: 177-178° C.

FTIR: 1659,1628 cm⁻¹

¹H-NMR (DMSO-d6) δ=8.21 (d, 1H); 8.17 (d, 1H); 8.14 (d, 1H); 7.87 (d, 1H); 7.79 (t, 1H); 7.78 (t, 1H); 7.67 (t, 1H); 7.64 (t, 1H); 7.22 (s, 1H); 3.85 (t, 1H); 3.51 (t, 1H); 2.62 (t, 1H); 2.13 (t, 1H) ppm.

### Example 4

### 2-(10-Hydroxy-10H-anthracen-9-ylidene)-1-(4-methoxy-phenyl)-ethanone (Compound 4)

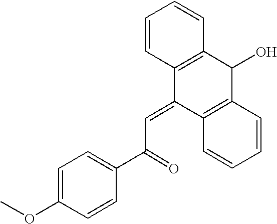

Melting point m.p.: 169-172° C.

FTIR: 3421,1638 cm⁻¹

¹H-NMR (DMSO-d6) δ=7.98 (d, 2H); 7.91 (d, 1H); 7.46 (t, 1H); 7.41 (t, 1H); 7.31 (t, 1H); 7.25 (d, 1H); 7.12 (s, 1H); 7.08 (t, 1H); 6.98 (d, 2H); 6.30 (d, 1H); 5.54 (d, 1H); 3.82 (s, 3H) ppm.

### Example 5

### 10-{2-[4-(4-Methoxy-phenyl)-piperazin-1-yl]-2-oxo-ethylidene}-10H-anthracen-9-one (Compound 5)

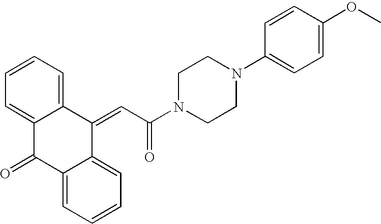

Melting point m.p.: 189-190° C.

¹H-NMR (DMSO-d6) δ=8.16-8.22 (m, 3H); 7.87 (d, 1H); 7.80 (t, 1H); 7.73 (t, 1H); 7.65 (t, 1H); 7.63 (t, 1H); 7.25 (s, 1H); 6.80 (m, 4H); 3.72 (m, 2H); 3.67 (s, 3H); 3.01 (m, 2H); 2.58 (m, 2H) ppm.

### Example 6

### 10-[2-(3,4-Dimethoxy-phenyl)-2-oxo-ethylidene]-9(10H)-anthracenone (Compound 6)

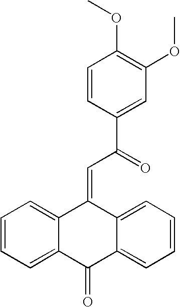

Melting point m.p.: 160° C.

FTIR: 1655,1639 cm⁻¹

¹H-NMR (DMSO-d6) δ=8.32 (d, 1H); 8.20 (d, 1H); 8.14 (d, 1H); 7.81 (t, 1H); 7.70 (s, 1H); 7.69 (t, 1H); 7.60 (d, 1H); 7.55 (t, 1H); 7.46-7.52 (m, 3H); 7.02 (d, 1H); 3.82 (s, 3H); 3.79 (s, 3H) ppm.

### Example 7

### 10-[2-(4-Methoxy-phenyl)-2-oxo-ethylidene]-10H-anthracen-9-one (Compound 7)

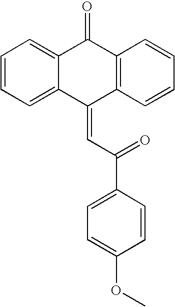

Melting point m.p.: 120-122° C.

FTIR 1647,1591 cm⁻¹

¹H-NMR (DMSO-d6) δ=8.32 (d, 1H); 8.20 (d, 1H); 8.14 (d, 1H); 7.96 (d, 2H); 7.83 (t, 1H); 7.72 (s, 1H); 7.69 (t, 1H); 7.46-7.56 (m, 3H); 7.03 (d, 2H); 3.81 (s, 3H) ppm.

### Example 8

### 10-[2-(2-Hydroxy-3,4-dimethoxy-phenyl)-2-oxo-ethylidene]-10H-anthracen-9-one (Compound 8)

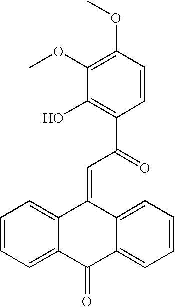

Melting point m.p.: 176-178° C.

FTIR 3030, 1655, 1615 cm⁻¹

¹H-NMR (DMSO-d6) δ=8.12 (d, 1H); 8.00 (d, 1H); 7.83 (d, 1H); 7.71 (t, 1H); 7.55 (t, 1H); 7.48 (t, 1H); 7.38-7.44 (m, 2H); 7.16 (d, 1H); 6.70 (d, 1H); 5.22 (s, 1H); 5.17 (s, 1H); 3.80 (s, 3H); 3.53 (s, 3H) ppm.

### Example 9

### 10-(2-Oxo-2-p-tolyl-ethylidene)-10H-anthracen-9-one (Compound 9)

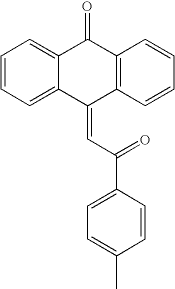

m.p. 129° C.;

FTIR 1645 cm⁻¹;

¹H-NMR (CDCl₃) δ=8.32 (dd, 1H), 8.23 (dd, 1H), 7.99 (d, 1H), 7.78, 7.15 (d, 2H), 7.72-7.66 (m, 1H), 7.62-7.55 (m, 2H), 7.44-7.38 (m, 1H), 7.30-7.14 (m, 1H), 7.20 (s, 1H), 3.11 (s, 3H);

### Example 10

### 10-[2-(4-Hydroxy-phenyl)-2-oxo-ethylidene]-10H-anthracen-9-one (Compound 10)

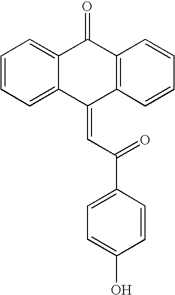

m.p. 211-212° C.;

FTIR 3321, 1665, 1638 cm⁻¹;

¹H-NMR (CDCl₃) δ=8.32 (dd, 1H), 8.23 (dd, 1H), 7.99 (d, 1H), 7.80, 6.77 (d, 2H), 7.72-7.67 (m, 1H), 7.62-7.56 (m, 2H), 7.44-7.39 (m, 1H), 7.32-7.29 (m, 1H), 7.17 (s, 1H), 6.14 (s, 1H):

### Example 11

### 10-(2-Oxo-2-thiophen-2-yl-ethylidene)-10H-anthracen-9-one (Compound 11)

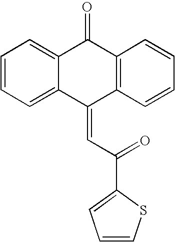

m.p. 126° C.;

FTIR 1658,1623 cm⁻¹;

¹H-NMR (CDCl₃) δ=8.32 (dd, 1H), 8.24 (dd, 1H), 7.97 (d, 1H), 7.74-7.68 (m, 2H), 7.63-7.58 (m, 2H), 7.48-7.44 (m, 2H), 7.39-7.35 (m, 1H), 7.19 (s, 1H), 6.98-6.96 (m, 1H);

### Example 12

### 10-[2-(3-Hydroxy-phenyl)-2-oxo-ethylidene]-10H-anthracen-9-one (Compound 12)

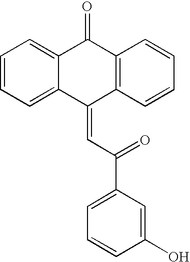

m.p. 185° C.;

FTIR 1661,1634 cm⁻¹;

¹H-NMR (CDCl₃) δ=8.35 (dd, 1H), 8.17 (dd, 1H), 7.92 (d, 1H), 7.65-7.61 (m, 1H), 7.55-7.52 (m, 1H), 7.48 (d, 1H), 7.39-7.31 (m, 3H), 7.26-7.22 (m, 1H), 7.18-7.14 (m, 1H), 7.12 (s, 1H), 6.94-6.92 (m, 1H), 4.98 (s, 1H);

### Example 13

### 10-[2-(3-Methoxy-phenyl)-2-oxo-ethylidene]-10H-anthracen-9-one (Compound 13)

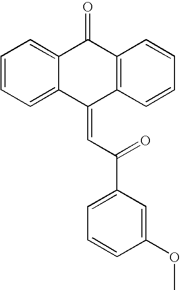

m.p. 129-130° C.

FTIR 1665 cm⁻¹;

¹H-NMR (CDCl₃) δ=8.32 (dd, 1H), 8.23 (dd, 1H), 7.99 (d, 1H), 7.72-7.68 (m, 1H), 7.62-7.58 (m, 1H), 7.54 (dd, 1H), 7.46-7.40 (m, 3H), 7.31-7.28 (m, 1H), 7.26-7.24 (m, 1H), 7.20 (s, 1H), 7.07-7.04 (m, 1H), 3.78 (s, 3H);

### Example 14

### (10-Oxo-10H-anthracen-9-ylidene)-acetic acid, 2,6-dimethoxy-phenyl ester (Compound 14)

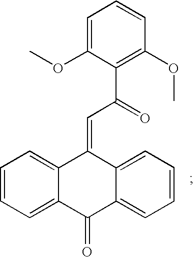

m.p. 172° C.;

FTIR 1722,1662 cm⁻¹;

¹H-NMR (CDCl₃) δ=8.28-8.23 (m, 2H), 7.95 (d, 1H), 7.68-7.64 (m, 1H), 7.60-7.54 (m, 3H), 7.17 (t, 1H), 6.96 (s, 1H), 6.65 (d, 2H), 3.87 (s, 6H);

### Example 15

### (10-Oxo-10H-anthracen-9-ylidene)-acetic acid methyl ester (Compound 15)

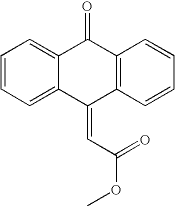

m.p. 113° C.

FTIR 1721,1661 cm⁻¹;

¹H-NMR (CDCl₃) δ=8.27-8.24 (m, 2H), 7.85-7.80 (m, 2H), 7.66-7.62 (m, 1H), 7.60-7.54 (m, 3H), 6.66 (s, 1H), 3.79 (s, 3H);

### Example 16

### 10-[2-Oxo-2-(3,4,5-trimethoxy-phenyl)-ethylidene]-10H-anthracen-9-one (Compound 16)

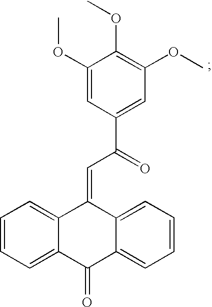

m.p. 151-152° C.

FTIR 1658 cm⁻¹;

¹H-NMR (CDCl₃) δ=8.30 (dd, 1H), 8.22 (dd, 1H), 7.98 (d, 1H), 7.72-7.60 (m, 1H), 7.49 (d, 1H), 7.44-7.40 (m, 1H), 7.12 (s, 1H), 7.08 (s, 2H), 3.85 (s, 3H), 3.72 (s, 6H);

### Example 17

### (10-Oxo-10H-anthracen-9-ylidene)-acetic acid (Compound 17)

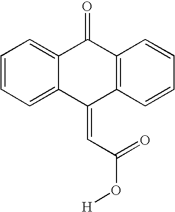

m.p. 180-182° C.

FTIR: 1684,1663 cm⁻¹;

¹H-NMR (DMSO-d6) δ=8.05-8.03 (m, 2H), 7.71-7.69 (m, 1H), 7.65-7.63 (m, 2H), 7.59-7.57 (dd, 1H), 7.50-7.44 (m, 2H), 7.07 (s, 1H).

### Example 18

### 10-(2-Oxo-2-phenyl-ethylidene)-10H-anthracen-9-one (Compound 18)

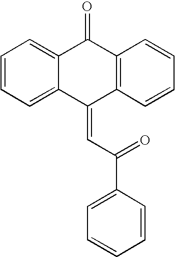

melting point: 128° C.

¹H-NMR ¹H-NMR (DMSO-d6) δ=8.33 (d, 1H), 8.20 (d, 1H), 8.13 (d, 1H), 7.99 (d, 2H), 7.83 (t, 1H), 7.74 (s, 1H), 7.70 (t, 1H), 7.67 (t, 1H), 7.47-7.56 (m, 5H).

The compounds 19-43 can be prepared in accordance with the general scheme 1.

### II) Inhibition of the Polymerization of Tubulin

a) In an in vitro test, the compounds 1, 6, 7, 9, 10, 12, 13, 16 and 18 according to the invention were tested for inhibition of the polymerization of bovine tubulin (D. M. Bollag et al. Cancer Res. 1995, 55: 2325-2333). In this test, tubulin purified by polymerization and depolymerization cycles is used, which is polymerized by addition of GTP and heating.

Table 1 states the %-inhibition values and Table 2 states the EC₅₀ values of polymerization inhibition of tubulin with 30% associated proteins (MAPs).

TABLE 1

| Verbindung | Inhibition at 10 μg/ml [%] |
|---|---|
| 1 | 98.2, 83.0, 86.9, 79.7 |
| 6 | 68.9, 86.1, 99.0 |
| 7 | 99.1, 100.0, 100.0 |
| 9 | 98.3, 99.8, 100.0, 100.0 |
| 10 | 85.0, 94.8, 98.4, 98.6 |
| 12 | 83.1, 95.0, 92.7, 86.9 |
| 13 | 72.6, 85.9, 87.3, 93.7 |
| 16 | 65.0, 65.5, 61.9 |
| 18 | 54.9, 69.9, 79.8, 71.0 |

TABLE 2

| Verbindung | EC50 [μg/mL] |
|---|---|
| 1 | 7.15/8.02 |
| 7 | 1.38/1.50 |
| 9 | 0.49 |
| 10 | 4.87 |

b) In an in vitro test, the compounds 1, 2, 4 and 7 were tested for inhibition of the polymerization of porcine brain tubulin. In this test, tubulin purified by polymerization and depolymerization cycles is used (Shelanski et al. Proc. Natl. Acad. Sci. U.S.A. 1973, 70, 765-768 modified according to Vater et al. Acta Histochem. Suppl. 1986, 33, 123-129), which is polymerized by addition of GTP and heating to 37° C. In addition to tubulin, the protein used comprises about 15% of associated proteins (MAPs). The measurement values were determined turbidimetrically according to Gaskin et al. J. Mol. Biol. 89 (1974) 737-755.

Table 3 states the EC₅₀ value of the polymerization inhibition.

TABLE 3

| Compound | EC50 [μg/mL] |
|---|---|
| 1 | 3.0 |
| 2 | 2.3 |
| 4 | 1.2 |
| 7 | 0.5 |

### III) Inhibition of Kinesin Eg5

In an in vitro test, compound 1 was examined for inhibition of Eg5 activity [Kodama, T. et al. J. Biochem. 99: 1465-1472 (1986)].

Here, the recombinant kinesins Eg5₁₃₋₄₃₇ (Table 3) and Eg5₁₋₄₀₆ (Table 4) were used.

Tables 4 and 5 state the EC₅₀ values of Eg5 inhibition for the compound 1 according to the invention.

TABLE 4

| Compound | EC50 [μg/mL] |
|---|---|
| 1 | 2.37/2.48/1.87/2.02 |

TABLE 5

| Compound | EC50 [μg/mL] |
|---|---|
| 1 | 12.7 |

### IV) Inhibition of Kinesin KIF5A

In an in vitro test, compound 1 was examined for inhibition of KIF5A ATPase activity. Here, use was made of complete recombinant KIF5A (Niclas J, Navone F, Hom-Booher N, Vale R D Neuron 1994, 12:1059-1072).

The activity of the microtubulus-activatable ATPase was determined by end-point assay. For tubulin isolation and preparation of the microtubuli see citations in Example II) b).

Table 6 states the EC₅₀ value of the ATPase inhibition of KIF5A by compound 1 according to the invention.

TABLE 6

| Compound | EC50 [μg/mL] |
|---|---|
| 1 | 21.2 |

### V) Antiproliferative Effect on Various Tumor Cell Lines

The antiproliferative activity of the compounds 1, 2, 4, 6, 7, 9, 10, 11, 12 and 13 according to the invention was investigated in a proliferation test performed on established tumor cell lines (D. A. Scuderio et al. Cancer Res. 1988, 48: 4827-4833).

The test used determines the cellular dehydrogenase activity and makes it possible to determine cell viability and determine cell number indirectly.

The cell lines used are the human cervical carcinoma cell line KB/HeLa (ATCC CCL17), the ovarian adenocarcinoma cell line SKOV-3 (ATCC HTB77), the human glioblastoma cell line SF-268 (NCI-503138) and the lung carcinoma cell line NCI-H460 (NCI-503473). In addition, an RKOp27 cell system was used for investigating the cell cycle-specific effect of the substance (M. Schmidt et al. Oncogene 2000, 19(20): 2423-9).

RKO is a human colon carcinoma line in which expression of the cell cycle inhibitor p27^{kiP1} can be induced using the ecdysone expression system and lead to cell cycle arrest specifically in G2.

A substance which acts nonspecifically inhibits proliferation independently of whether the RKO cell is arrested in G1 or G2 or not. By contrast, cell cycle-specific substances, such as, for example, tubulin inhibitors, are only cytotoxic when cells are not arrested and the cell cycle is passed through.

Table 7 shows the cytotoxic or growth-inhibiting activity of the described compounds with and without expression of p27^{kiP1}. When p27^{kiP1} was in the induced state, the tested compounds did not exhibit any cytotoxic activity. The results show that the compounds according to the invention exhibit very potent inhibition of the proliferation of selected tumor cell lines.

TABLE 7

| | KB/HeLa | NCI-H460 | SF-268 | SK-OV-3 | RKOP27 | RKOP27^{ind} |
|---|---|---|---|---|---|---|
| | EC50 | EC50 | EC50 | EC50 | EC50 | EC50 |
| Verbindung | [μg/mL] | [μg/mL] | [μg/mL] | [μg/mL] | [μg/mL] | [μg/mL] |
| 1 | 0.091 | 0.146 | 0.076 | 0.053 | 0.083 | >3.16 |
| 2 | 0.248 | 0.931 | 0.331 | 0.416 | 0.802 | >3.16 |
| 4 | 0.181 | 0.203 | 0.227 | 0.171 | 0.108 | >3.16 |
| 6 | 0.458 | 0.290 | n.d. | n.d. | 0.240 | >3.16 |
| 7 | 0.020 | 0.025 | 0.020 | 0.020 | 0.027 | >3.16 |
| 9 | 0.20 | 0.11 | 0.20 | 0.13 | 0.09 | >3.16 |
| 10 | 2.65 | 1.34 | n.d. | n.d. | 1.38 | >3.16 |
| 11 | 2.65 | 1.05 | n.d. | n.d. | n.d. | >3.16 |
| 12 | 1.40 | 0.61 | 2.22 | 3.03 | 0.63 | >3.16 |
| 13 | 1.89 | 1.16 | n.d. | n.d. | 1.10 | >3.16 |

### VI) Antiproliferative Effect on Various Multidrug-Resistant (MDR) Tumor Cell Lines

For further characterization, the compounds 6 and 7 according to the invention were investigated with regard to multidrug-resistant (MDR) cell lines as compared with the nonresistant wild-type cell lines.

The cell lines which were investigated are the murine cell line L1210 (ATCC CCL219), the acute myeloid leukaemia cell line LT12 (De Vries et al.; University Rotterdam) and the resistant lines L1210/VCR (Asta Medica Laboratory) and LT12/mdr (De Vries et al.; University Rotterdam).

The murine P388 cell line (methylcholanthrene-induced lymphoid neoplasm) [R. Supino et al.; Inst. Nazionale per lo Studio e las cura die Tumori; Milan)] and the doxorubicin-resistant P388 cell line (NSC-123127) were also included as test systems.

Table 8 shows the corresponding XTT data.

TABLE 8

| | LT12 | LT12mdr | L1210 | L1210VCR | P388 | P388ADR |
|---|---|---|---|---|---|---|
| | IC50 | IC50 | IC50 | IC50 | IC50 | IC50 |
| Compound | [μg/mL] | [μg/mL] | [μg/mL] | [μg/mL] | [μg/mL] | [μg/mL] |
| 6 | 0.10/0.17 | 0.10/0.23 | 0.32/0.35 | 0.26/0.54 | 0.35/0.39 | 0.25/0.49 |
| 7 | 0.015/0.019 | 0.015/0.020 | 0.016/0.017 | 0.017/0.018 | 0.014/0.015 | 0.016/0.018 |

### VII) Cell Cycle Analysis

The cell cycle comprises the progress of the cell from one cell generation to the next.

During the resetting phase (G0) and the presynthetic phase (G1), the cell possesses a diploid set of chromosomes (2c). In the synthetic phase (S), the quantity of DNA is increased by replication. The S phase ends when the premitotic phase (G2M), in which the cell possesses a reduplicated complement of chromosomes (4c) and a doubled content of DNA, is reached. In the subsequent mitotic phase (M), which is of short duration, the reduplicated chromosomes are uniformly apportioned between two daughter cells, which then in each case once again possess a diploid content of DNA and are in the G01 phase, which means that the cell cycle can begin afresh.

For the cell cycle analysis, KB/HeLa cells (ATCC CCL17) were treated with different concentrations (0.1-1000 nM) of the test substances at 37° C. for 24 hours.

The percentage of cells arrested in the G2/M phase of the cell cycle after treatment with compounds according to the invention and selected reference substances is shown in Table 9 below. The results were analysed using special analytical software (ModFit™).

TABLE 9

| | EC50 [nM] |
|---|---|
| Compound | (50% cells in G2/M) |
| 1 | 0.131/0.303 |
| Paclitaxel | 26.9 |

1. An anthracene compound according to formula (I) 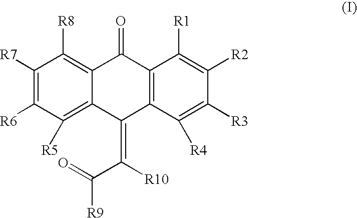
in which:
substituent X is independently selected from the group consisting of O, S, NR11, N—OR12, geminally attached hydrogen and hydroxyl;
substituent Y is independently selected from the group consisting of O, S, NR13, N—OR14;
substituents R1, R2, R3, R4, R5, R6, R7, R8 independently of one another are independently selected from the group consisting of hydrogen, unsubstituted or substituted alkyl, unsubstituted or substituted cycloalkyl, unsubstituted or substituted cycloalkyl-alkyl, unsubstituted or substituted heterocyclyl, unsubstituted or substituted heterocyclyl-alkyl, unsubstituted or substituted aryl, unsubstituted or substituted aryl-alkyl, unsubstituted or substituted heteroaryl, unsubstituted or substituted heteroaryl-alkyl, amino, mono-alkylamino, di-alkylamino, halogen, —F, —Cl, —Br, —I, alkyl substituted by one or more fluorine atoms, trifluoromethyl, cyano, straight-chain or branched cyano-alkyl, carbonyl, carboxyl, —COOH, alkoxy-carbonyl, carboxy-alkyl, alkoxycarbonyl-alkyl, hydroxyl, alkoxy, aryl-alkoxy, benzyloxy, heteroaryl-alkoxy, alkoxycarbonylamino, alkoxycarbonylamino-alkyl;
substituent R9 is independently selected from the group consisting of unsubstituted or substituted alkyl, unsubstituted or substituted cycloalkyl, unsubstituted or substituted cycloalkyl-alkyl, unsubstituted or substituted heterocyclyl, unsubstituted or substituted heterocyclyl-alkyl, unsubstituted or substituted aryl, unsubstituted or substituted aryl-alkyl, unsubstituted or substituted heteroaryl, unsubstituted or substituted heteroaryl-alkyl, —OR15, —NR16R17;
substituent R10 is independently selected from the group consisting of hydrogen, unsubstituted or substituted alkyl, unsubstituted or substituted aryl, unsubstituted or substituted aryl-alkyl, unsubstituted or substituted heteroaryl, unsubstituted or substituted heteroaryl-alkyl;
substituents R11, R12, R13, R14, R15, R16, R17 independently of one another are independently selected from the group consisting of hydrogen, unsubstituted or substituted alkyl, unsubstituted or substituted cycloalkyl, unsubstituted or substituted cycloalkyl-alkyl, unsubstituted or substituted heterocyclyl, unsubstituted or substituted heterocyclyl-alkyl, unsubstituted or substituted aryl, unsubstituted or substituted aryl-alkyl, unsubstituted or substituted heteroaryl, unsubstituted or substituted heteroaryl-alkyl;
with the proviso that the following compounds are excluded:
(i) the compound having the Chemical Abstract Service (CAS) Registry No. 262378-52-9 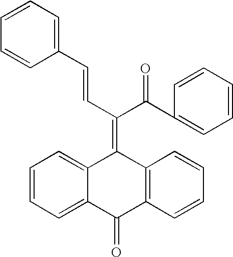
(ii) the compound having the Chemical Abstract Service (CAS) Registry No. 121747-08-8 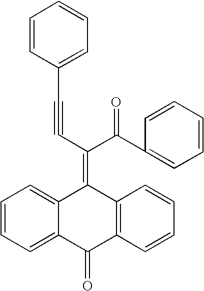
(iii) the compound having the Chemical Abstract Service (CAS) Registry No. 63370-49-0 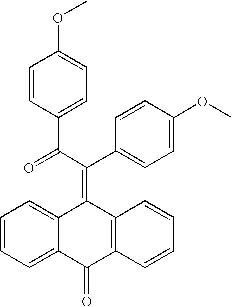
(iv) the compound having the Chemical Abstract Service (CAS) Registry No. 19661-62-2 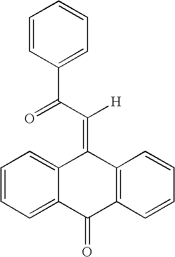
(v) the compound having the Chemical Abstract Service (CAS) Registry No. 17665-75-7 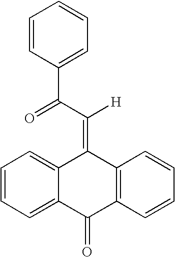
(vi) the compound having the Chemical Abstract Service (CAS) Registry No. 83498-19-5 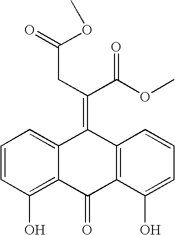
(vii) the compound having the Chemical Abstract Service (CAS) Registry No. 42866-43-3 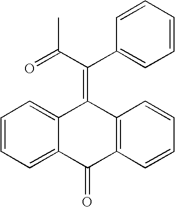
(viii) the compound having the Beilstein Registry No. 3095926 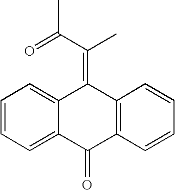
(ix) the compound having the Chemical Abstract Service (CAS) Registry No. 42866-49-9

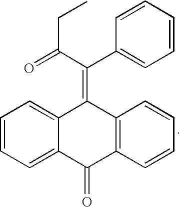

2. An anthracene compound according to claim 1 in which:
substituent X is independently selected from the group consisting of: O, S, NH, geminally attached hydrogen and hydroxyl;
substituent Y is independently O or S;
substituents R1, R2, R3, R4, R5, R6, R7, R8 are independently hydrogen, fluorine, chlorine, bromine, iodine, hydroxyl, amino, nitro, cyano, methyl, trifluoromethyl;
substituent R9 is independently selected from the group consisting of: unsubstituted or substituted heterocyclyl, unsubstituted or substituted aryl, unsubstituted or substituted heteroaryl, —OR15, —NR16R17;
substituent R10 is independently hydrogen, alkyl.

3. An anthracene compound according to claim 1 in which:
substituent R9 is independently selected from the group consisting of: 3,4-dimethoxy-phenyl; 3,4-dihydroxy-phenyl; 4-methoxy-thiophen-2-yl, thiomorpholin-4-yl; 4-methoxy-phenyl; 4-(4-methoxy-phenyl)-piperazin-1-yl, 2-hydroxy-3,4-dimethoxy-phenyl; 4-methylphenyl; 4-hydroxyphenyl; thiophen-2-yl; 3-hydroxyphenyl; 3-methoxy-phenyl; 2,6-dimethoxy-phenyl; 3,4,5-trimethoxy-phenyl; hydroxyl; methoxy; phenyl; 4-chlorophenyl; 4-bromophenyl; 4-fluorophenyl; 3-chlorophenyl; 3-bromophenyl; 3-fluorophenyl; 4-fluoro-3-hydroxy-phenyl.

4. An anthracene compound according to claim 1, selected from the group consisting of:
10-[2-(3,4-dihydroxy-phenyl)-2-oxo-ethylidene]-10H-anthracen-9-one (Compound 1): 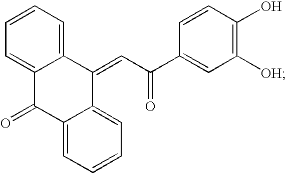
10-[2-(4-methoxy-thiophen-2-yl)-2-oxo-ethylidene]-10H-anthracen-9-one (Compound 2): 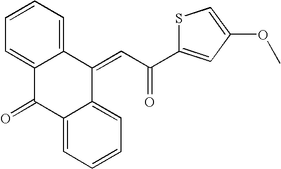
10-(2-oxo-2-thiomorpholin-4-ylethylidene)-10H-anthracen-9-one (Compound 3): 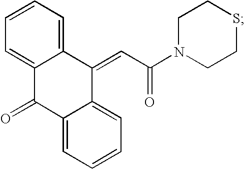
2-(10-hydroxy-10H-anthracen-9-ylidene)-1-(4-methoxy-phenyl)-ethanone (Compound 4): 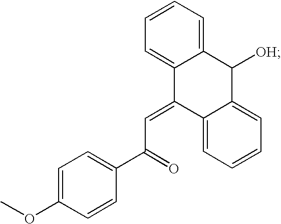
10-{2-[4-(4-methoxy-phenyl)-piperazin-1-yl]-2-oxo-ethylidene}-10H-anthracen-9-one (Compound 5): 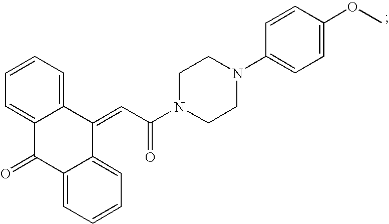
10-[2-(3,4-dimethoxy-phenyl)-2-oxo-ethylidene]-9(10H)-anthracenone (Compound 6): 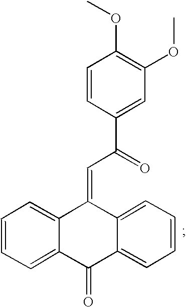
10-[2-(4-methoxy-phenyl)-2-oxo-ethylidene]-10H-anthracen-9-one (Compound 7): 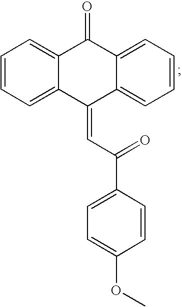
10-[2-(2-hydroxy-3,4-dimethoxy-phenyl)-2-oxo-ethylidene]-10H-anthracen-9-one (Compound 8) 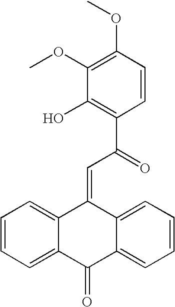
10-(2-oxo-2-p-tolyl-ethylidene)-10H-anthracen-9-one (Compound 9) 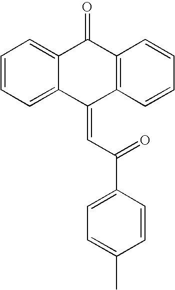
10-[2-(4-hydroxy-phenyl)-2-oxo-ethylidene]-10H-anthracen-9-one (Compound 10) 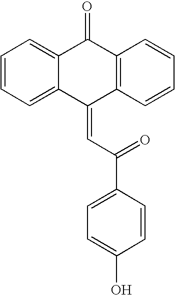
10-(2-oxo-2-thiophen-2-yl-ethylidene)-10H-anthracen-9-one (Compound 11) 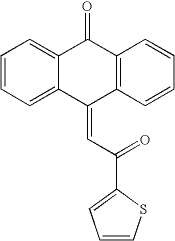
10-[2-(3-hydroxy-phenyl)-2-oxo-ethylidene]-10H-anthracen-9-one (Compound 12) 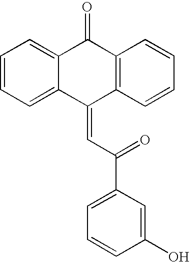
10-[2-(3-methoxy-phenyl)-2-oxo-ethylidene]-10H-anthracen-9-one (Compound 13) 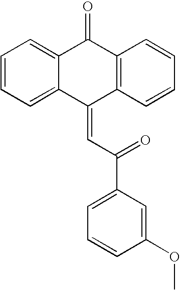
(10-oxo-10H-anthracen-9-ylidene)-acetic acid, 2,6-dimethoxy-phenyl ester (Compound 14) 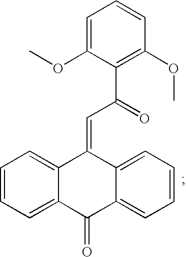
(10-oxo-10H-anthracen-9-ylidene)-acetic acid methyl ester (Compound 15) 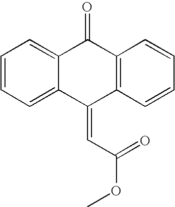
10-[2-oxo-2-(3,4,5-trimethoxy-phenyl)-ethylidene]-10H-anthracen-9-one (Compound 16) 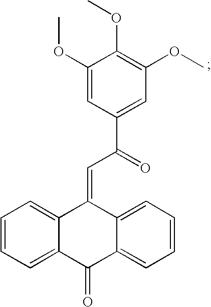
(10-oxo-10H-anthracen-9-ylidene)-acetic acid (Compound 17) 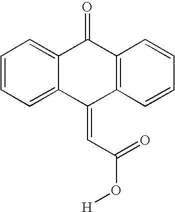
10-(2-oxo-2-phenyl-ethylidene)-10H-anthracen-9-one (Compound 18) 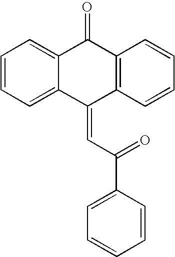
10-[2-(4-chloro-phenyl)-2-oxo-ethylidene]-10H-anthracen-9-one (Compound 19) 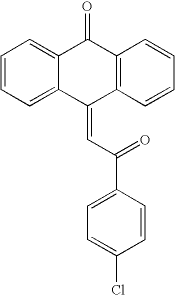
10-[2-(4-bromo-phenyl)-2-oxo-ethylidene]-10H-anthracen-9-one (Compound 20) 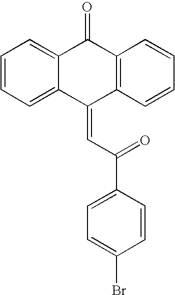
10-[2-(3-chloro-phenyl)-2-oxo-ethylidene]-10H-anthracen-9-one (Compound 21) 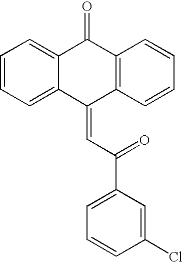
10-[2-(3-bromo-phenyl)-2-oxo-ethylidene]-10H-anthracen-9-one (Compound 22) 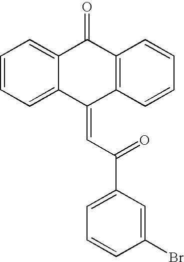
10-[2-(4-fluoro-phenyl)-2-oxo-ethylidene]-10H-anthracen-9-one (Compound 23) 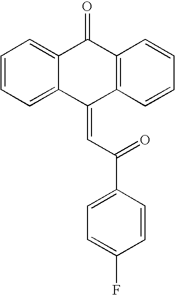
10-[2-(3-fluoro-phenyl)-2-oxo-ethylidene]-10H-anthracen-9-one (Compound 24) 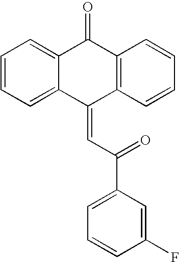
10-[2-(4-fluoro-3-hydroxy-phenyl)2-oxo-ethylidene]-10H-anthracen-9-one (Compound 25) 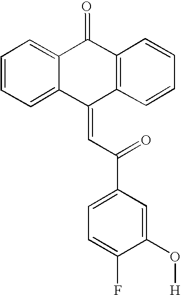
1-(3,4-dihydroxy-phenyl)-2-(10-thioxo-10H-anthracen-9-ylidene)-ethanone (Compound 26): 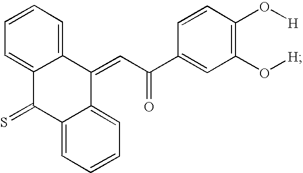
1-(3,4-dihydroxy-phenyl)-2-(10-imino-10H-anthracen-9-ylidene)-ethanone (Compound 27): 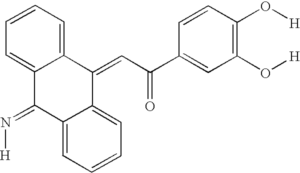
1-(3-hydroxy-phenyl)-2-(10-thioxo-10H-anthracen-9-ylidene)-ethanone (Compound 28): 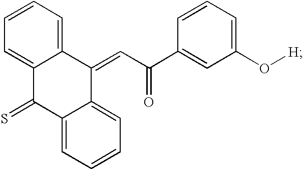
1-(3-hydroxy-phenyl)-2-(10-imino-10H-anthracen-9-ylidene)-ethanone (Compound 29): 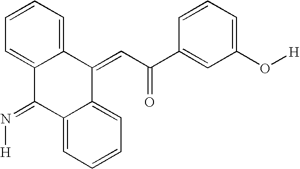
1-(4-fluoro-3-hydroxy-phenyl)-2-(10-thioxo-10H-anthracen-9-ylidene)-ethanone (Compound 30): 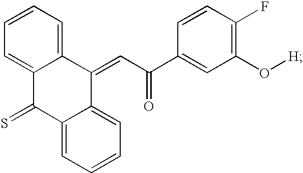
1-(4-fluoro-3-hydroxy-phenyl)-2-(10-imino-10H-anthracen-9-ylidene)-ethanone (Compound 31): 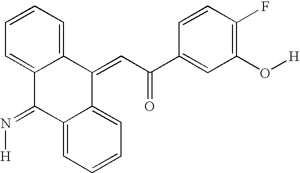
1,4-dichloro-10-[2-(3-hydroxy-phenyl)-2-oxo-ethylidene]-10H-anthracen-9-one (Compound 32) 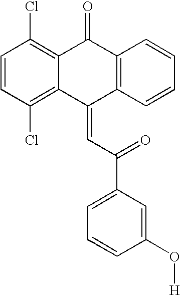
1,2-dichloro-10-[2-(3-hydroxy-phenyl)-2-oxo-ethylidene]-10H-anthracen-9-one (Compound 33) 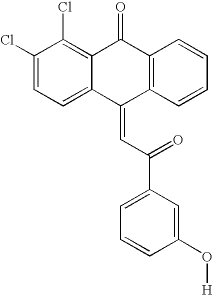
3,4-dichloro-10-[2-(3-hydroxy-phenyl)-2-oxo-ethylidene]-10H-anthracen-9-one (Compound 34) 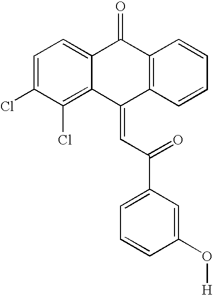
1,2-methyl-10-[2-(3-hydroxy-phenyl)-2-oxo-ethylidene]-10H-anthracen-9-one (Compound 35) 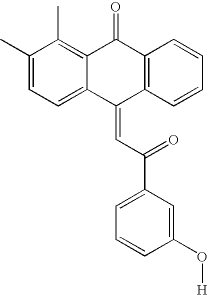
3-chloro-10-[2-(3-hydroxy-phenyl)-2-oxo-ethylidene]-10H-anthracen-9-one (Compound 36) 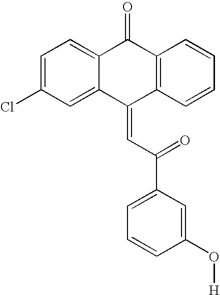
3-bromo-10-[2-(3-hydroxy-phenyl)-2-oxo-ethylidene]-10H-anthracen-9-one (Compound 37) 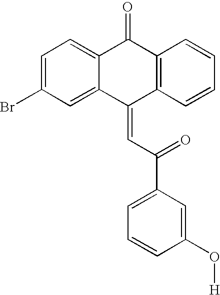
10-[2-(3-hydroxy-phenyl)-2-oxo-ethylidene]-3-methyl-10H-anthracen-9-one (Compound 38) 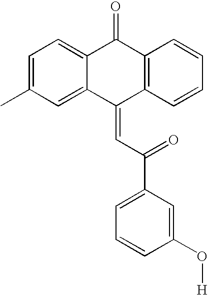
1,4-dichloro-10-[2-(3,4-dihydroxy-phenyl)-2-oxo-ethylidene]-10H-anthracen-9-one (Compound 39) 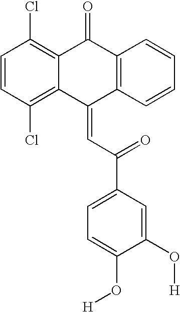
3,4-dimethyl-10-[2-(3-hydroxy-phenyl)-2-oxo-ethylidene]-10H-anthracen-9-one (Compound 40) 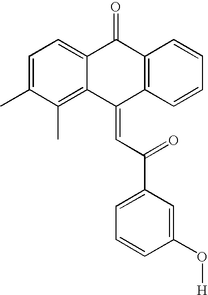
2-[2-chloro-10-thioxo-10H-anthracen-9-ylidene]-1-(3-hydroxy-phenyl)-ethanone (Compound 41) 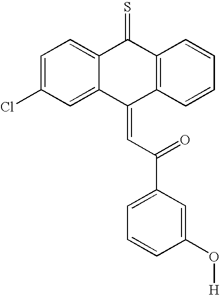
3-chloro-10-[2-(3,4-dihydroxy-phenyl)-2-oxo-ethylidene]-10H-anthracen-9-one (Compound 42) 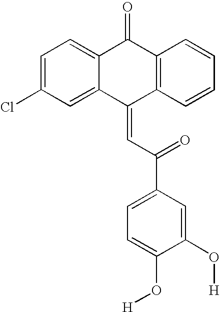
2-[2-chloro-10-imino-10H-anthracen-9-ylidene]-1-(3-hydroxy-phenyl)-ethanone and (Compound 43)

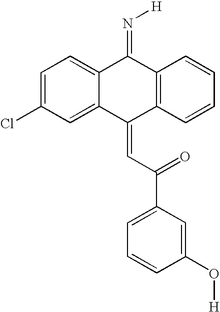

5. A pharmaceutical composition comprising a pharmaceutically active amount of at least one compound according to claim 1.

6. A composition according to claim 5 where the at least one compound is present in a unit dose of from 0.001 mg to 100 mg per kg of body weight of a patient.

7. A composition according to claim 5 where the composition further comprises at least one pharmaceutically acceptable carrier and/or auxiliary.

8. A composition according to claim 5, wherein the composition further comprises at least one additional pharmacologically active substance.

9. A composition according to claim 8, comprising at least one additional pharmacologically active substance selected from the group consisting of: DNA topoisomerase I and/or II inhibitors, DNA intercalators, alkylating agents, microtubuli destabilizers, hormone and/or growth factor receptor agonists and/or antagonists, inhibitors of signal transduction, antibodies against growth factors and their receptors, kinase inhibitors, and antimetabolites.

10. A composition according to claim 8, comprising at least one additional pharmacologically active substance selected from the group consisting of: actinomycin D, aminoglutethimide, asparaginase, avastin, azathioprine, BCNU (carmustine), bleomycin, busulfan, carboplatin, CCNU (lomustine), chlorambucil, cisplatin, colaspase, cyclophosphamide, cytarabine, dactinomycin, daunorubicin, diethylstilbestrol, doxorubicin (adriamycin), DTIC (dacarbacin), epirubicin, erbitux, erythrohydroxynonyladenine, ethynyloestradiol, etoposide, fludarabine phosphate, fluoxymesterone, flutamide, gemcitabine, Gleevec/Glivec, Herceptin, hexamethylmelamine, hydroxyurea, hydroxyprogesterone caproate, idarubicin, ifosfamide, interferon, iressa, irinotecan, L-asparaginase, leucovorin, mechlorethamine, medroxyprogesterone acetate, megestrol acetate, melphalan, mesna, methotrexate, mitomycin C, mitotane, mitoxantrone, N-phosphonoacetyl-L-aspartate (PALA), oxaliplatin, pentostatin, plicamycin, prednisolone, prednisone, procarbazine, raloxifen, rapamycin, semustine, sorafenib, streptozocin, tamoxifen, tarceva, taxotere, teniposide, testosterone propionate, thioguanine, thiotepa, topotecan, trimethylmelamine, uridine, vinblastine, vincristine, vindesine, vinorelbine, 2′,2′-difluorodeoxycytidine, 5-fluorodeoxyuridine monophosphate, 5-azacytidine cladribine, 5-fluorodeoxyuridine, 5-fluorouarcil (5-FU), and 6-mercaptopurine.

11. A method for treating a benign or malignant tumor disorder, comprising administering at least one anthracene compound according to formula (II) to a mammal in need thereof: 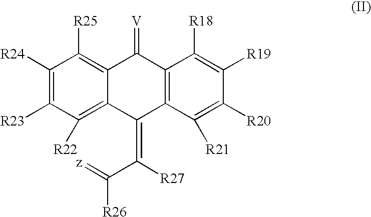
in which:
substituent V is independently selected from the group consisting of: O, S, NR28, N—OR29, geminally attached hydrogen and hydroxyl;
substituent Z is independently selected from the group consisting of: O, S, NR30, N—OR31;
substituents R18, R19, R20, R21, R22, R23, R24, R25 independently of one another are independently selected from the group consisting of: hydrogen, unsubstituted or substituted alkyl, unsubstituted or substituted cycloalkyl, unsubstituted or substituted cycloalkyl-alkyl, unsubstituted or substituted heterocyclyl, unsubstituted or substituted heterocyclyl-alkyl, unsubstituted or substituted aryl, unsubstituted or substituted aryl-alkyl, unsubstituted or substituted heteroaryl, unsubstituted or substituted heteroaryl-alkyl, amino, mono-alkylamino, di-alkylamino, halogen, —F, —Cl, —Br, —I, alkyl substituted by one or more fluorine atoms, trifluoromethyl, cyano, straight-chain or branched cyano-alkyl, carbonyl, carboxyl, —COOH, alkoxycarbonyl, carboxy-alkyl, alkoxycarbonyl-alkyl, hydroxyl, alkoxy, aryl-alkoxy, benzyloxy, heteroaryl-alkoxy, alkoxycarbonylamino, alkoxycarbonylamino-alkyl;
substituent R26 is independently selected from the group consisting of: unsubstituted or substituted alkyl, unsubstituted or substituted cycloalkyl, unsubstituted or substituted cycloalkyl-alkyl, unsubstituted or substituted heterocyclyl, unsubstituted or substituted heterocyclyl-alkyl, unsubstituted or substituted aryl, unsubstituted or substituted aryl-alkyl, unsubstituted or substituted heteroaryl, unsubstituted or substituted heteroaryl-alkyl, —OR32, —NR33R34;
substituent R27 is independently selected from the group consisting of: hydrogen, unsubstituted or substituted alkyl, unsubstituted or substituted aryl, unsubstituted or substituted aryl-alkyl, unsubstituted or substituted heteroaryl, unsubstituted or substituted heteroaryl-alkyl;
substituents R28, R29, R30, R31, R32, R33, R34 independently of one another are independently selected from the group consisting of: hydrogen, unsubstituted or substituted alkyl, unsubstituted or substituted cycloalkyl, unsubstituted or substituted cycloalkyl-alkyl, unsubstituted or substituted heterocyclyl, unsubstituted or substituted heterocyclyl-alkyl, unsubstituted or substituted aryl, unsubstituted or substituted aryl-alkyl, unsubstituted or substituted heteroaryl, unsubstituted or substituted heteroaryl-alkyl.

12. A method for treating a benign or malignant tumor disorder according to claim 11, comprising administering at least one anthracene compound according to the formula (II) in which:
substituent V is independently selected from the group consisting of: O, S, NH, geminally attached hydrogen and hydroxyl;
substituent Z is independently O or S;
substituents R18, R19, R20, R21, R22, R23, R24, R25 are independently hydrogen, fluorine, chlorine, bromine, iodine, hydroxyl, amino, nitro, cyano, methyl, trifluoromethyl;
substituent R26 is independently selected from the group consisting of: unsubstituted or substituted heterocyclyl, unsubstituted or substituted aryl, unsubstituted or substituted heteroaryl, —OR32, —NR33R34;
substituent R27 is independently hydrogen, alkyl.

13. A method for treating a benign or malignant tumor disorder according to claim 11, comprising administering at least one anthracene compound according to the formula (II) in which:
substituent R27 is independently selected from the group consisting of: 3,4-dimethoxy-phenyl; 3,4-dihydroxy-phenyl; 4-methoxy-thiophen-2-yl, thiomorpholin-4-yl; 4-methoxy-phenyl; 4-(4-methoxy-phenyl)-piperazin-1-yl, 2-hydroxy-3,4-dimethoxy-phenyl, 4-methylphenyl; 4-hydroxyphenyl; thiophen-2-yl; 3-hydroxyphenyl; 3-methoxy-phenyl; 2,6-dimethoxy-phenyl, 3,4,5-trimethoxy-phenyl, hydroxyl; methoxy; phenyl; 4-chlorophenyl; 4-bromophenyl; 4-fluorophenyl; 3-chlorophenyl; 3-bromophenyl; 3-fluorophenyl; 4-fluoro-3-hydroxy-phenyl.

14. A method for the treatment or prophylaxis of physiological and/or pathophysiological conditions which can be treated by inhibiting the tubulin polymerization and/or by inhibiting microtubuli-based motor proteins comprising administering at least one compound according to claim 4 to a mammal in need thereof.

15. A method for the treatment or prophylaxis of physiological and/or pathophysiological conditions which can be treated by inhibiting the tubulin polymerization and/or by inhibiting microtubuli-based motor proteins comprising administering at least one compound according to formula (II) to a mammal in need thereof: 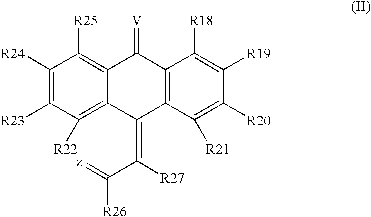
in which:
substituent V is independently selected from the group consisting of: O, S, NR28, N—OR29, geminally attached hydrogen and hydroxyl;
substituent Z is independently selected from the group consisting of: O, S, NR30, N—OR31;
substituents R18, R19, R20, R21, R22, R23, R24, R25 independently of one another are independently selected from the group consisting of: hydrogen, unsubstituted or substituted alkyl, unsubstituted or substituted cycloalkyl, unsubstituted or substituted cycloalkyl-alkyl, unsubstituted or substituted heterocyclyl, unsubstituted or substituted heterocyclyl-alkyl, unsubstituted or substituted aryl, unsubstituted or substituted aryl-alkyl, unsubstituted or substituted heteroaryl, unsubstituted or substituted heteroaryl-alkyl, amino, mono-alkylamino, di-alkylamino, halogen, —F, —Cl, —Br, —I, alkyl substituted by one or more fluorine atoms, trifluoromethyl, cyano, straight-chain or branched cyano-alkyl, carbonyl, carboxyl, —COOH, alkoxycarbonyl, carboxy-alkyl, alkoxycarbonyl-alkyl, hydroxyl, alkoxy, aryl-alkoxy, benzyloxy, heteroaryl-alkoxy, alkoxycarbonylamino, alkoxycarbonylamino-alkyl;
substituent R26 is independently selected from the group consisting of: unsubstituted or substituted alkyl, unsubstituted or substituted cycloalkyl, unsubstituted or substituted cycloalkyl-alkyl, unsubstituted or substituted heterocyclyl, unsubstituted or substituted heterocyclyl-alkyl, unsubstituted or substituted aryl, unsubstituted or substituted aryl-alkyl, unsubstituted or substituted heteroaryl, unsubstituted or substituted heteroaryl-alkyl, —OR32, —NR33R34;
substituent R27 is independently selected from the group consisting of: hydrogen, unsubstituted or substituted alkyl, unsubstituted or substituted aryl, unsubstituted or substituted aryl-alkyl, unsubstituted or substituted heteroaryl, unsubstituted or substituted heteroaryl-alkyl;
substituents R28, R29, R30, R31, R32, R33, R34 independently of one another are independently selected from the group consisting of: hydrogen, unsubstituted or substituted alkyl, unsubstituted or substituted cycloalkyl, unsubstituted or substituted cycloalkyl-alkyl, unsubstituted or substituted heterocyclyl, unsubstituted or substituted heterocyclyl-alkyl, unsubstituted or substituted aryl, unsubstituted or substituted aryl-alkyl, unsubstituted or substituted heteroaryl, unsubstituted or substituted heteroaryl-alkyl.

16. The method according to claim 15 where the method is a method for the treatment or prophylaxis of physiological and/or pathophysiological conditions which can be treated by inhibiting microtubuli-based motor proteins, wherein the microtubuli-based motor proteins are selected from the group consisting of: kinesins, dyneins, kinesin superfamily (KIF) protein, N-1 kinesins, KIF5A, KIF5B, KIF5C, N-2 kinesins, Eg5 (KIF11, KSP), BimC (KIF8), N-3 kinesins, KIF1A, KIF1B, KIF1C, KIF13A, KIF13B, KIF14, KIF16A, KIF16B, N-4 kinesins, KIF3A, KIF3B, KIF3C, KIF17, N-5 kinesins, KIF4, KIF4A, KIF4B, KIF21A, KIF21B, N-6 kinesins, KIF23 (MKLP1, CHO1), KIF20A (Rab6-KIF), KIF20B (KIpMPP1), MKLP2, N-7 kinesins, KIF10 (CENP-E), N-8 kinesins, KIF18A, KIF18B, KIF22 (Kid), KIF19A, KIF19B, N-9 kinesins, KIF12, N-10 kinesins, KIF15 (HkIp2), N-11 kinesins, KIF24, KIF25 (KNSL3), KIF26A, KIF26B, M-kinesins, KIF2A (KIF2), KIF2B, KIF2C (MCAK), C-1 kinesins, KIFC1, C-2 kinesins, KIFC2, KIFC3, KIF6, KIF7, and KIF9.

17. The method according to claim 11, wherein said method is a method for the treatment or prophylaxis of malignant tumors, benign tumors, solid tumors, sarcomas, carcinomas, hyperproliferative disorders, carcinoids, Ewing sarcomas, Kaposi sarcomas, brain tumors, tumors originating from the brain and/or the nervous system and/or the meninges, gliomas, neuroblastomas, stomach cancer, kidney cancer, kidney cell carcinomas, prostate cancer, prostate carcinomas, connective tissue tumors, soft tissue sarcomas, pancreas tumors, liver tumors, head tumors, neck tumors, oesophageal cancer, thyroid cancer, osteosarcomas, retinoblastomas, thymoma, testicular cancer, lung cancer, bronchial carcinomas, breast cancer, mamma carcinomas, intestinal cancer, colorectal tumors, colon carcinomas, rectum carcinomas, gynaecological tumors, ovary tumors/ovarian tumors, uterine cancer, cervical cancer, cervix carcinomas, cancer of body of uterus, corpus carcinomas, endometrial carcinomas, urinary bladder cancer, bladder cancer, skin cancer, basaliomas, spinaliomas, melanomas, intraocular melanomas, leukaemias, chronic leukaemias, acute leukaemias and/or lymphomas.

18. A kit comprising a pharmacologically active amount of at least one compound according to claim 4 and a pharmacologically active amount of at least one further pharmacologically active substance.

19. A kit comprising a pharmacologically active amount of at least one compound according to formula (II) and a pharmacologically active amount of at least one further pharmacologically active substance: 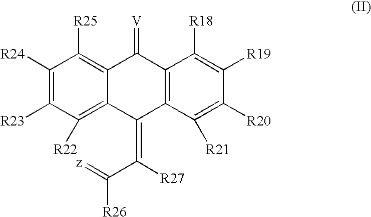
in which:
substituent V is independently selected from the group consisting of: O, S, NR28, N—OR29, geminally attached hydrogen and hydroxyl;
substituent Z is independently selected from the group consisting of: O, S, NR30, N—OR31;
substituents R18, R19, R20, R21, R22, R23, R24, R25 independently of one another are independently selected from the group consisting of: hydrogen, unsubstituted or substituted alkyl, unsubstituted or substituted cycloalkyl, unsubstituted or substituted cycloalkyl-alkyl, unsubstituted or substituted heterocyclyl, unsubstituted or substituted heterocyclyl-alkyl, unsubstituted or substituted aryl, unsubstituted or substituted aryl-alkyl, unsubstituted or substituted heteroaryl, unsubstituted or substituted heteroaryl-alkyl, amino, mono-alkylamino, di-alkylamino, halogen, —F, —Cl, —Br, —I, alkyl substituted by one or more fluorine atoms, trifluoromethyl, cyano, straight-chain or branched cyano-alkyl, carbonyl, carboxyl, —COOH, alkoxycarbonyl, carboxy-alkyl, alkoxycarbonyl-alkyl, hydroxyl, alkoxy, aryl-alkoxy, benzyloxy, heteroaryl-alkoxy, alkoxycarbonylamino, alkoxycarbonylamino-alkyl;
substituent R26 is independently selected from the group consisting of: unsubstituted or substituted alkyl, unsubstituted or substituted cycloalkyl, unsubstituted or substituted cycloalkyl-alkyl, unsubstituted or substituted heterocyclyl, unsubstituted or substituted heterocyclyl-alkyl, unsubstituted or substituted aryl, unsubstituted or substituted aryl-alkyl, unsubstituted or substituted heteroaryl, unsubstituted or substituted heteroaryl-alkyl, —OR32, —NR33R34;
substituent R27 is independently selected from the group consisting of: hydrogen, unsubstituted or substituted alkyl, unsubstituted or substituted aryl, unsubstituted or substituted aryl-alkyl, unsubstituted or substituted heteroaryl, unsubstituted or substituted heteroaryl-alkyl;
substituents R28, R29, R30, R31, R32, R33, R34 independently of one another are independently selected from the group consisting of: hydrogen, unsubstituted or substituted alkyl, unsubstituted or substituted cycloalkyl, unsubstituted or substituted cycloalkyl-alkyl, unsubstituted or substituted heterocyclyl, unsubstituted or substituted heterocyclyl-alkyl, unsubstituted or substituted aryl, unsubstituted or substituted aryl-alkyl, unsubstituted or substituted heteroaryl, unsubstituted or substituted heteroaryl-alkyl.
